(19) United States
(12) Patent Application Publication
(10) Pub. No.: US 2012/0253210 A1
(43) Pub. Date: Oct. 4, 2012

(54) Cuff of Sphygmomanometer

(75) Inventors: Chisato Uesaka, Kyoto-shi (JP); Yukiya Sawanoi, Nara-shi (JP)

(73) Assignee: OMRON HEALTHCARE CO., LTD., Kyoto (JP)

(21) Appl. No.: 13/404,160

(22) Filed: Feb. 24, 2012

(30) Foreign Application Priority Data
Mar. 28, 2011 (JP) 2011-069937

(51) Int. Cl. A61B 5/022

(52) U.S. Cl. 600/499

(57) ABSTRACT

A cuff of a sphygmomanometer includes a fluid bladder connected to a main body of the sphygmomanometer via a tube and a band-shaped wrapping body that encloses the fluid bladder. The band-shaped wrapping body includes two longitudinal sides and two lateral sides. A weight section is provided along a longitudinal side of the band-shaped wrapping body that faces a peripheral end of a user's arm when the cuff is mounted to the arm in accordance with an expected method of use.

### BACKGROUND OF INVENTION

The present invention relates to a cuff of a sphygmomanometer used for measuring blood pressure information by wearing on the upper arm and a sphygmomanometer equipped therewith.

Measuring blood pressure information is absolutely critical in understanding a health condition. In recent years, this is not just limited to measuring the maximum blood pressure, the minimum blood pressure, and the like, where such utility is widely recognized as a major indicator contributing to risk analysis of, for example, cardiovascular diseases such as stroke, heart failure, and myocardial infraction, but tests have also been undertaken to also capture the cardiac load, arterial sclerosis level, or the like.

A sphygmomanometer is a device to measure the blood pressure information, and further utilization in fields such as early detection, prevention, and medical treatment of circulatory diseases are expected. Note that the blood pressure information widely includes various information of the circulatory system such as various indicators showing the maximum blood pressure, minimum blood pressure, mean blood pressure, pulse wave, pulse, and arterial sclerosis level.

Particularly, even in the medical field, there is a tendency to emphasize the measurement of blood pressure information at home because blood pressure information can be continually measured at home at the same time daily over a long period of time under stable circumstances. For example, it is proven that the measurement of the maximum blood pressure and the minimum blood pressure at home (hereinafter refer to as the measurement of blood pressure) is extremely useful for predicting a cardiovascular disease, and blood pressure monitors for home use have been widely used recently.

Commonly, a sphygmomanometer cuff (hereinafter, also referred to as simply the cuff) is used for measuring blood pressure information. Here, the cuff indicates a band-shaped or cylindrical-shaped structural material that includes a fluid bag with an inner space, and it can be worn on a part of a biological body. Further, it is used to measure blood pressure information by injecting a gas, fluid, or the like into the inner space to inflate the fluid bag for applying pressure on an artery.

In order to measure the blood pressure information with a higher degree of accuracy, the cuff needs to be worn properly on the upper arm. Normally, because the fluid bag contained in the cuff inflates the most at the center section in the wrapped direction while the cuff is worn on the upper arm, the applied section contributes the most for applying pressure on the artery. As a result, sphygmomanometers are most likely designed based on the premise that the applied portion of the cuff is placed directly above the artery that runs in the upper arm. Accordingly, if the wearing position and direction of the cuff relation to the upper arm differs from the expectation, pressure cannot be applied to the artery sufficiently and the accuracy of the measurement of the blood pressure information deteriorates.

In the blood pressure monitor for home use described above, most of the users are not healthcare professionals, so a user does not necessarily wear the cuff appropriately. Accordingly, various designs have been devised in the past so as to wear the cuff properly on the arm.

For example, JP Unexamined Utility Model Application No. 60-81506 (Patent Document 1) discloses a blood pressure monitor structured by integrating the cuff and the blood pressure monitor body and providing a recess section for positioning so that the cuff can be fastened on the arm elbow to help in wearing the cuff properly.

Further, JP Unexamined Patent Application Publication No. 2007-275483 (Patent Document 2) discloses a blood pressure monitor cuff structured by providing a protruding tongue that temporarily holds that should be tucked by the arm elbow, underarm, or the like so as to help in wearing the cuff properly.

Furthermore, JP Unexamined Patent Application Publication No. 2007-275484 (Patent Document 3) discloses a blood pressure monitor cuff structured by providing a positioning marking in a specified spot of the fluid bag and also providing a window section in the cuff to be worn on the upper arm so that the positioning marking is viewable from the outside to help in wearing the cuff properly.

### SUMMARY OF INVENTION

However, the blood pressure monitor cuffs disclosed in the above mentioned patent documents are all structured by providing a viewable characteristic section such as a positioning recessed area, marking, or protruding tongue for temporarily holding, and therefore, the cuff can be properly worn only after a user understands the meaning of the characteristic section provided. Accordingly, there are some cases that a user cannot understand the meaning and cannot wear the cuff properly by utilizing it, so these solutions are not necessarily achieved completely in the fundamental significance.

Therefore, one or more embodiments of the present invention provide a cuff of a sphygmomanometer in which a user can properly wear the cuff naturally without paying a special caution/attention, and also provide a sphygmomanometer equipped therewith.

One or more embodiments of the present invention provide a cuff of a sphygmomanometer which comprises: a fluid bladder connected to a main body of the sphygmomanometer via a tube; and a band-shaped wrapping body that encloses the fluid bladder. The band-shaped wrapping body comprises two longitudinal sides and two lateral sides. A weight section is provided along a longitudinal side of the band-shaped wrapping body that faces a peripheral end of user's arm when the cuff is mounted to the arm in accordance with an expected method of use.

In the cuff of the sphygmomanometer according to one or more embodiments of the present invention, the weight section is provided in a plurality of numbers at regular intervals.

In the cuff of the sphygmomanometer according to one or more embodiments of the present invention, the cuff is formed in a cylindrical shape having a hollow part such that the user's arm can be inserted into the hollow part.

One or more embodiments of the present invention provide a cuff of sphygmomanometer that comprises: a fluid bladder connected to a main body of the sphygmomanometer via a tube; and a band-shaped wrapping body that encloses the fluid bladder. A cylindrical shell for inserting a user's arm is rotatably connected to the main body of the sphygmomanometer and the band-shaped wrapping body is mounted in a hole of the shell. A weight section is provided at a portion of the band-shaped wrapping body that contacts a vertical bottom position of the cylindrical shell when a user's arm is inserted into the hollow part of the shell in accordance with an expected method of use.

One or more embodiments of the present invention provide a cuff of sphygmomanometer which comprises: a fluid bladder connected to a main body of the sphygmomanometer via a tube; and a band-shaped wrapping body that encloses the fluid bladder. A cylindrical shell is rotatably connected to the main body of the sphygmomanometer. The shell is provided with a handle at a vertical top portion thereof, and the band-shaped wrapping body is mounted in a hole of the shell to form a hollow part for inserting a user's arm therein. The band-shaped wrapping body is provided with a weight section at a bottom position of the cylindrical shell that is opposite to the position of the handle in the shell when a user's arm is inserted into the hollow part of the shell in accordance with an expected method of use.

One or more embodiments of the present invention provide a sphygmomanometer which comprises a main body and a cylindrical shell having a hole. The shell is rotatably connected to the main body. A cuff is stored in the hole of the shell. The cuff forms a hollow part for inserting a user's arm therein. The cuffs provided with a weight section at a vertical bottom position of a user's arm when the user's arm is inserted into the hollow part of the cuff stored in the hole of the shell in accordance with an expected method of use.

One or more embodiments of the present invention provide a sphygmomanometer which comprises: a main body and a cylindrical shell that is rotatably connected to the main body. The shell is provided with a handle at a vertical top position thereof. A cuff is stored in a hole of the shell to form a hollow part for inserting a user's arm therein. The cuff is provided with a weight section at a vertical bottom position of the shell that is opposite to the handle of the shell when the user's arm is inserted into the hollow part of the shell in accordance with an expected method of use.

According to one or more embodiments of the present invention, a sphygmomanometer cuff can be provided in which a user can properly wear the cuff naturally without paying special attention, and a sphygmomanometer equipped therewith can also be provided.

### BRIEF DESCRIPTION OF THE DRAWINGS

FIG. 1 is a schematic perspective view of a blood pressure monitor cuff according to the first embodiment of the present invention and a blood pressure monitor equipped therewith;

FIG. 2 is a diagram illustrating a configuration of a functional block of the blood pressure monitor illustrated in FIG. 1;

FIG. 3 is a schematic expanded view of the blood pressure monitor cuff illustrated in FIG. 1;

FIG. 4 is a schematic cross-sectional view of the blood pressure monitor ci rff illustrated in FIG. 1;

FIG. 5 is a diagram illustrating a state when the blood pressure monitor cuff illustrated in FIG. 1 is worn on an upper arm;

FIG. 6 is a diagram of an operational flow of a control unit of the blood pressure monitor illustrated in FIG. 1;

FIG. 7 is a schematic expanded view of the blood pressure monitor cuff for a modified example in accordance with the first embodiment of the present invention;

FIG. 8 is a schematic perspective view of a blood pressure monitor cuff according to a second embodiment of the present invention;

FIG. 9 is a schematic expanded view of the blood pressure monitor cuff illustrated in FIG. 8;

FIG. 10 is a schematic cross-sectional view of the blood pressure monitor cuff illustrated in FIG. 8;

FIG. 11 is a diagram illustrating a state when the blood pressure monitor cuff illustrated in FIG. 8 is worn on the upper arm;

FIG. 12 is a schematic perspective view of a blood pressure monitor cuff according to a third embodiment of the present invention and a blood pressure monitor equipped therewith;

FIG. 13 is a diagram illustrating a configuration of a functional block of the blood pressure monitor illustrated in FIG. 12;

FIG. 14 is a schematic cross-sectional view of the blood pressure monitor cuff illustrated in FIG. 12;

FIG. 15 is a diagram illustrating a state when the blood pressure monitor cuff illustrated in FIG. 12 is worn on the upper arm;

FIG. 16 is a diagram of an operational flow of a control unit of the blood pressure monitor illustrated in FIG. 12;

FIG. 17 is a schematic perspective view of a blood pressure monitor cuff for a modified example according to the third embodiment of the present invention;

FIG. 18 is a schematic perspective view of a blood pressure monitor cuff according to a fourth embodiment of the present invention;

FIG. 19 is a schematic cross-sectional view of the blood pressure monitor cuff illustrated in FIG. 18;

FIG. 20 is a diagram illustrating a state when the blood pressure monitor cuff illustrated in FIG. 18 is worn on the upper arm;

FIG. 21 is a schematic perspective view of a blood pressure monitor cuff that relates to a first modified example according to the fourth embodiment of the present invention;

FIG. 22 is a schematic perspective view of a blood pressure monitor cuff that relates to a second modified example according to the fourth embodiment of the present invention;

FIG. 23 is a schematic perspective view of a blood pressure monitor cuff that relates to a third modified example according to the fourth embodiment of the present invention;

FIG. 24 is a schematic perspective view of a blood pressure monitor cuff that relates to a fourth modified example according to the fourth embodiment of the present invention;

FIG. 25 is a schematic perspective view of a blood pressure monitor cuff that relates to a fifth modified example according to the fourth embodiment of the present invention;

FIG. 26 is a schematic perspective view of a blood pressure monitor cuff that relates to a sixth modified example according to the fourth embodiment of the present invention;

FIG. 27 is a schematic perspective view of a blood pressure monitor cuff in the fifth embodiment of the present invention; and

FIG. 28 is a schematic perspective view of a blood pressure monitor cuff that relates to a modified example according to the fifth embodiment of the present invention.

### DETAILED DESCRIPTION OF INVENTION

Embodiments of the present invention will be described in detail hereinafter with reference to drawings. In the embodiments described hereinafter, descriptions will be given by illustrating a blood pressure monitor cuff that is used by wearing on the arm and a blood pressure monitor equipped therewith as a sphygmomanometer cuff and a sphygmomanometer equipped therewith. In addition, in the embodiments illustrated below, the same notation is given for the same or common parts in drawings, and the descriptions thereof are not repeated.

### The First Embodiment

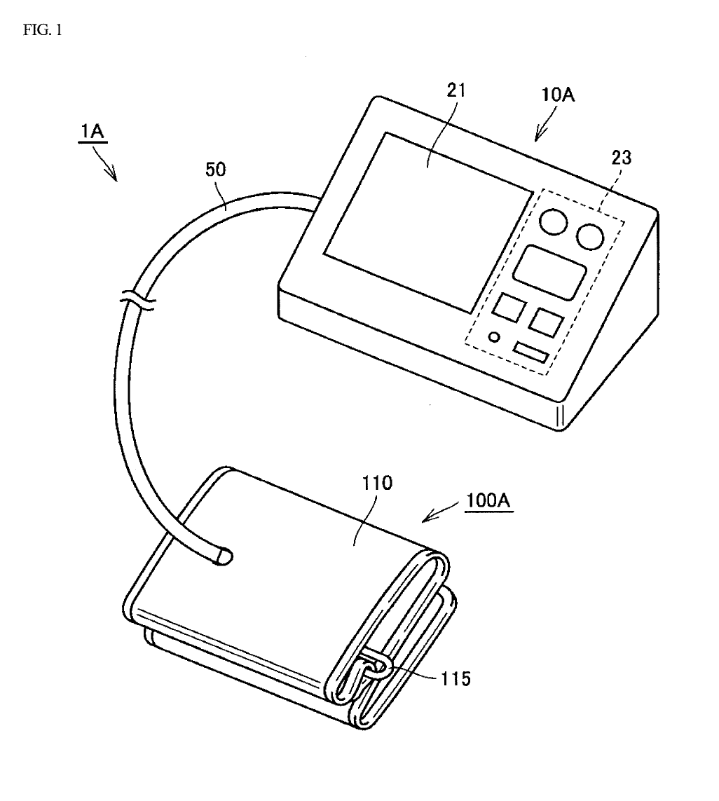

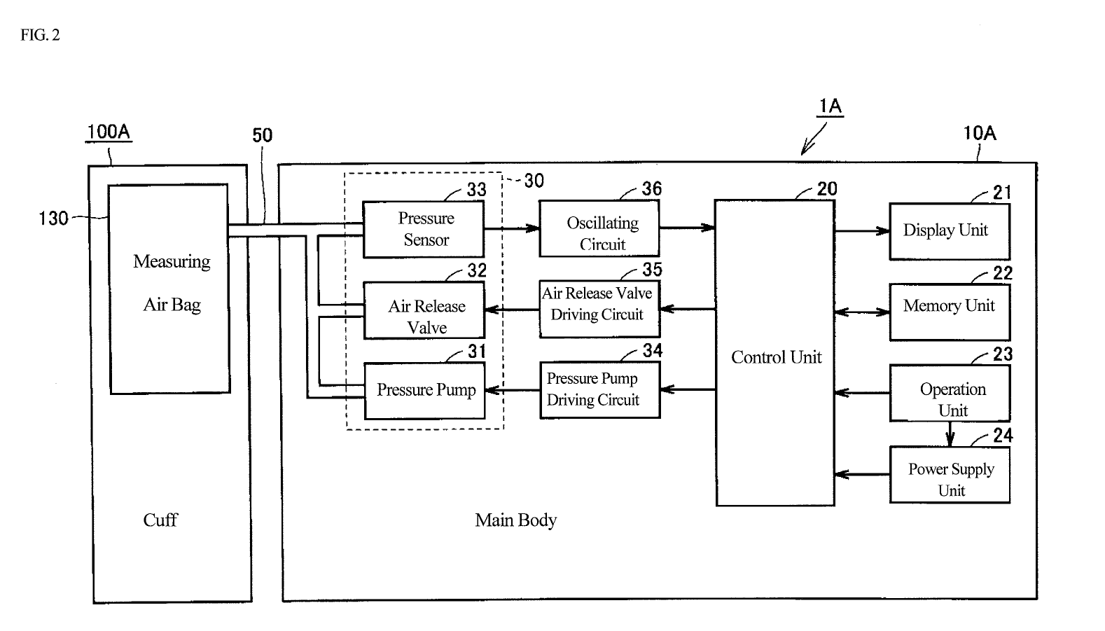

FIG. 1 is a schematic perspective view of a blood pressure monitor cuff according to a first embodiment of the present invention and a blood pressure monitor equipped therewith. Further, FIG. 2 is a diagram illustrating a configuration of a functional block of the blood pressure monitor illustrated in FIG. 1. To begin with, a description will be given regarding a structure of the exterior view and a configuration of a functional block of a blood pressure monitor 1A according to the first embodiment of the present invention with reference to FIG. 1 and FIG. 2.

As illustrated in FIG. 1, the blood pressure monitor 1A in the present embodiment is provided with a main body 10A, a off 100A, and an air tube 50. The main body 10A has a box casing, and on the top surface thereof, a display unit 21 and an operation unit 23 are provided. The main body 10A is used by placing on a placement surface such as a table or the like at the time of measuring. The cuff 100A has primarily an outer cover 110 as a wrapping body, a ring member 115, and a measuring air bag 130 (refer to FIG. 2 through FIG. 4), and has a wrapping shape as a whole. The a cuff 100A is used by wrapping around the upper arm to put on at the time of measuring. The air tube 50 connects the main body 10A and the cuff 100A that are configured separately.

As illustrated in FIG. 2, the main body 10A has, in addition to the display unit 21 and the operation unit 23 described above, a control unit 20, a memory unit 22, a power supply unit 24, a pressure pump 31, an air release valve 32, a pressure sensor 33, a pressure pump driving circuit 34, an air release valve driving circuit 35, and an oscillating circuit 36. The pressure pump 31, air release valve 32, and pressure sensor 33 correspond to a measurement air system component 30 that is provided in the blood pressure monitor 1A, and particularly the pressure pump 31 and the air release valve 32 correspond to the inflate-deflate mechanism to inflate and deflate the measuring air bag 130.

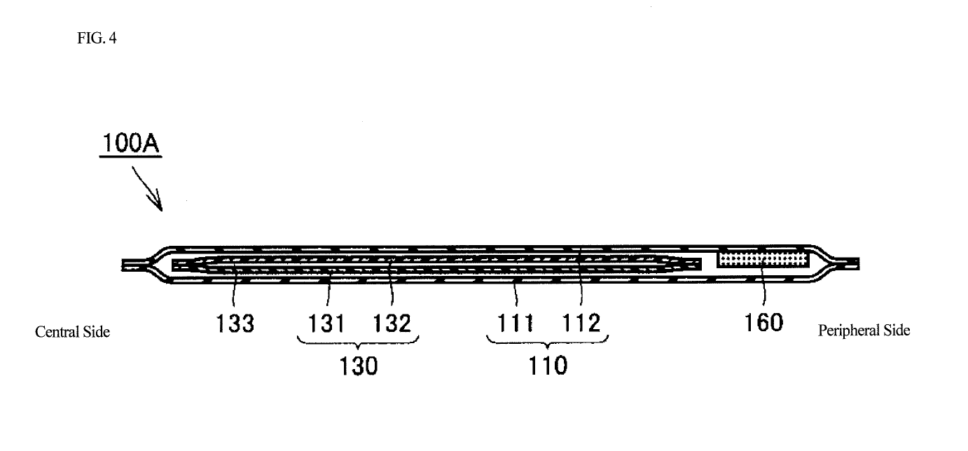

The measuring air bag 130 is a fluid bag in order to apply compression on the upper arm while worn, and an inflate-deflate space 133 (refer to FIG. 4) is provided as the inner space therein. The measuring air bag 130 is connected respectively to the pressure pump 31, air release valve 32, and pressure sensor 33 that are the measurement air system component 30 described above through the air tube 50 described above.

The control unit 20 is configured with, for example, a central processing unit (CPU) for means to control the entire blood pressure monitor 1A. The memory unit 22 is configured of, for example, read-only memory (ROM) and random-access memory (RAM) for means to store a program to execute the processing procedure for measuring the blood pressure by the control unit 20 or the like, and to store the results of the measurement and the like. The display unit 21 is configured with, for example, a liquid crystal display (LCD) for means to display the measurement results and the like. The operation unit 23 is means to accept an operation by a user and input an external command to the control unit 20 and the power supply unit 24. The power supply unit 24 is means to supply the power to the control unit as the power source.

The control unit 20 inputs a control signal in order to drive the pressure pump 31 and the air release valve 32 to the pressure pump driving circuit 34 and the air release valve driving circuit 35 respectively, and inputs the blood pressure level as the measured result into the memory unit 22 and the display unit 21. Further, the control unit 20 includes a blood pressure information acquisition unit (not illustrated) to acquire the blood pressure level of a user based on the pressure value detected by the pressure sensor 33, and the blood pressure level acquired by the blood pressure information acquisition unit is input to the memory unit 22 and the display unit 21 described above as the measured result. In addition, the blood pressure monitor 1A may have an output unit separately to output the blood pressure level as the measured result to an external device (for example a personal computer (PC), printer, and the like). As the output unit, for example, a serial communication line or devices to write to various recording media can be used.

The pressure pump driving circuit 34 controls the operation of the pressure pump 31 based on the control signal input from the control unit 20. The air release valve driving circuit 35 controls the opening-closing operation of the air release valve 32 based on a control signal input from the control unit 20. The pressure pump 31 is for applying pressure to the internal pressure (hereinafter, also referred to as “cuff pressure”) of the measuring air bag 130 by supplying air to the inflate-deflate space 133 of the measuring air bag 130, and the operation is controlled by the pressure pump driving circuit 34 described above. The air release valve 32 is for maintaining the internal pressure of the measuring air bag 130 and depressurizing the cuff pressure by releasing the inflate-deflate space 133 of the measuring air bag 130 to the outside, and the operation is controlled by the air release valve driving circuit 35 described above. The pressure sensor 33 is a capacitive-type sensor, and the capacitance varies according to the internal pressure of the measuring air bag 130. The oscillating circuit 36 generates an oscillation frequency signal in accordance with the capacitance of the pressure sensor 33, and inputs the generated signal to the control unit 20.

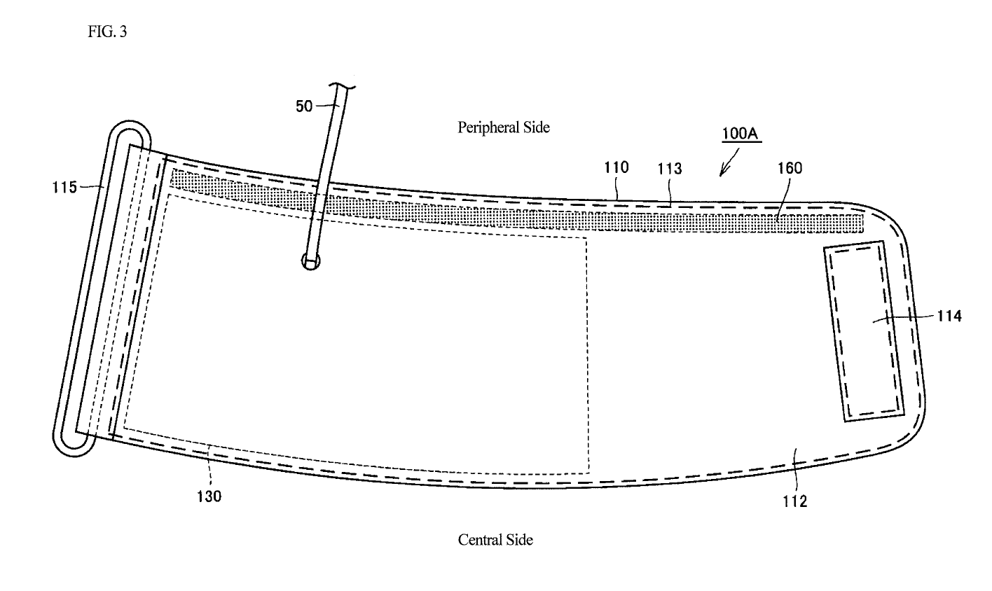

FIG. 3 is a schematic expanded view of the blood pressure monitor cuff illustrated in FIG. 1, and FIG. 4 is a schematic cross-sectional view. Next, a specific structure of the blood pressure monitor cuff 100A according to the present embodiment will be described with reference to FIG. 3 and FIG. 4.

As illustrated in FIG. 3 and FIG. 4, the blood pressure monitor cuff 100A according the present embodiment has a measuring air bag 130 that is connected to an air tube 50, and has an outer cover 110 as the wrapping body that contains the measuring air bag 130. In the state where the cuff 100A is expanded as illustrated in FIG. 3, the measuring air bag 130 has an external form that is substantially rectangular in a planar view, and an outer cover 110 has an external form that is substantially rectangular in a planar view that contains the measuring air bag 130.

As illustrated in FIG. 4, according to one or more embodiments of the present invention, the measuring air bag 130 is configured of a bag-like member formed by using a resin sheet. More specifically, the measuring air bag 130 has an inner sheet member 131 that is placed in the upper arm side when the cuff 100A is wrapped around the upper arm, and an outer sheet member 132 that is placed in the outer side of the inner sheet member 131 when the cuff 100A is wrapped around the upper arm, and has the inflate-deflate space 133 described above inside thereof. The measuring air bag 130 is formed in a bag like shape by laying the inner sheet member 131 and the outer sheet member 132 on top of each other and welding the peripheral edges thereof.

For the resin sheet that configures the measuring air bag 130, any material may be possible to use as long as it has elasticity and there is no air leakage from the inflate-deflate space 133 after welding. From such a standpoint, according to one or more embodiments of the present invention, a material made of, such as, an ethylene-vinyl acetate copolymer (EVA) resin, flexible polyvinyl chloride (PVC) resin, polyurethane (PU) resin, polyamide (PA) resin, or the like can be used.

Further, as illustrated in FIG. 4, an outer cover 110 has an inner cover member 111 that is placed on the upper arm side when the cuff 100A is wrapped around the upper arm, an outer cover member 112 that is placed on the outer side of the inner cover member 111 when the cuff 100A is wrapped around the upper arm, and a housing space to house the measuring air bag 130 described above or the like inside thereof. The outer cover 110 is formed in a bag like shape by laying the inner cover member 111 and the outer cover member 112 on top of each other and joining the peripheral edges thereof.

According to one or more embodiments of the present invention, the outer cover 110 has flexibility, and it is configured of a cloth made of a synthetic fiber such as a polyamide (PA) resin, a polyester resin, and the like. Further, in order to unite the inner cover member 111 and the outer cover member 112 described above, for example, welding, sewing, or the like are used. Here, the joining part is illustrated with the reference numeral 113 in FIG. 3. According to one or more embodiments of the present invention, the inner cover member 111 is configured of a member with excellent elasticity, and the outer cover member 112 is configured of a member with a rather less elasticity compared to the inner cover member 111 described above.

As illustrated in FIG. 3, a ring member 115 is attached to one end of the lengthwise direction of the outer cover 110. The ring member 115 functions as an assist tool when fixing the cuff 100A wrapping around the upper ami, and the cu ffA can be wrapped around the upper arm by inserting the other end of the lengthwise direction of the outer cover 110 into the ring member 115 and folding it back.

Further, as illustrated in FIG. 3, a hook-and-loop fastener 114 is provided on the outer circumference surface in the vicinity of the other end described above. The hook-and loop fastener 114 functions as a locking means in order to retain the state of wrapping the cuff 100A around the upper arm by locking the outer circumference surface of the outer cover 110 in the specified position in the state where the cuff 100A is wrapped around the upper arm.

In the blood pressure monitor cuff 100A in the present embodiment, as illustrated in FIG. 3 and FIG. 4, a weighted section 160 is provided in the vicinity of the peripheral side end part of the outer cover 110 that is arranged on the peripheral side of the upper arm while wearing. According to one or more embodiments of the present invention, the weighted section 160 is provided by extending along the lengthwise direction of the outer cover 110, and the weighted section 160 is configured with a deformable member that is able to deform in any shape.

The weighted section 160 contains a weight member that has a greater specific gravity than other members that configure the cuff 100A. For the weight member, in order to ensure the deformability described above, a liquid, a soft solid matter, aggregated solid particles, or a mixture of these is used, and according to one or more embodiments of the present invention, a liquid such as water, a molded product made of a soft metal, aggregated solid particles (powder) such as sand, metal, or the like, a mixture of these, or the like is used. In addition, when a liquid or aggregated solid particles is used as the weight member, according to one or more embodiments of the present invention these weight members are sealed in a sealed body.

In addition, a method of fixing the weighted section 160 to the outer cover 110 is not particularly restricted, and as illustrated in FIG. 4, the weighted section 160 may be arranged within the housing space of the outer cover 110 with this fixed onto the outer cover 110, or the weighted section 160 may even be arranged outside the outer cover 110 and this fixed onto the outer cover 110. Further, the weighted section 160 may be directly fixed to the outer cover 110, or a pocket unit may be provided in the outer cover 110 and it is arranged by inserting into the pocket.

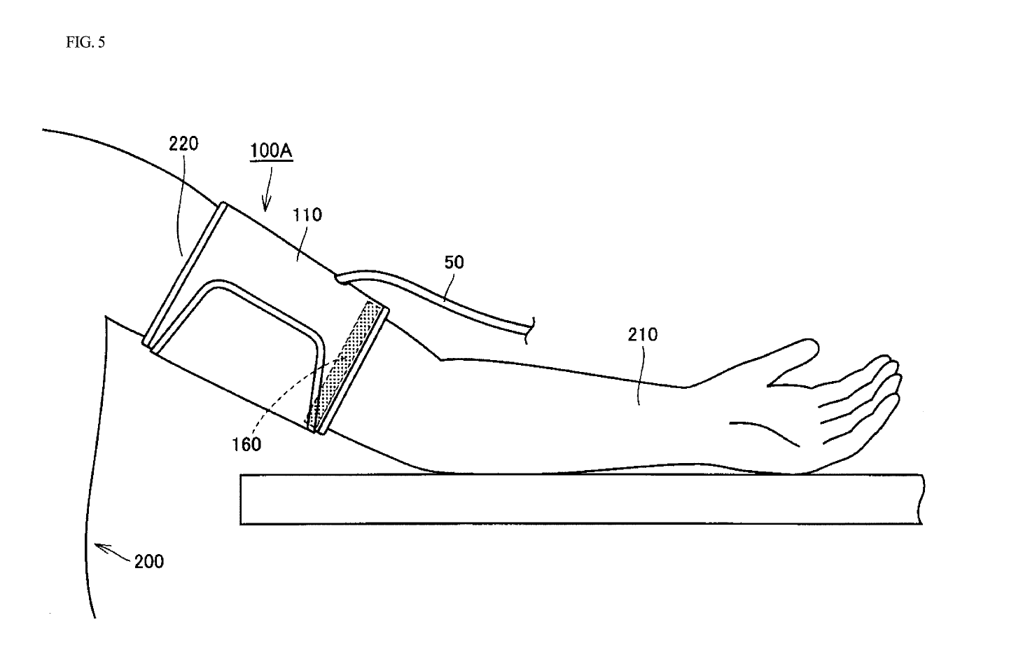

FIG. 5 is a diagram illustrating a state when the blood pressure monitor cuff illustrated in FIG. 1 is worn on the upper arm. Next, a description will be given with reference to FIG. 5 regarding the state when the blood pressure cuff 100A according to the present embodiment is actually worn on the upper arm for measuring the blood pressure.

As illustrated in FIG. 5, the blood pressure monitor cuff 100A according to the present embodiment is used while a user wears it on an upper arm 220 of, for example, the left arm 210. While wearing, the wrapped state of the cuff 100A is maintained by securing the portion that is folded back through the ring member 115 of the outer cover 110 by the hook-and-loop fastener 114 on the portion wrapped around the upper arm of the outer cover 110, and thereby the cuff 100A is worn by securing on the upper arm 220.

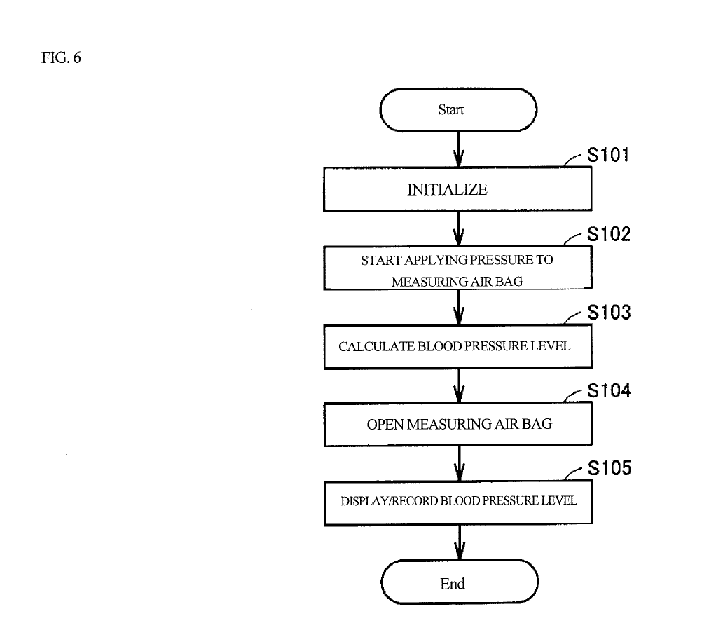

At that time, the peripheral side end part where the weighted section 160 of the outer cover 110 is provided is arranged on the elbow side position of the upper arm 220, and the central side end part where the weighted section 160 of the outer cover 110 is not provided is arranged in the shoulder side position of the upper arm 220. The blood pressure monitor 1A according to the present embodiment performs the blood pressure measurement while wearing by executing a processing procedure illustrated in FIG. 6, which is described later.

FIG. 6 is a flowchart illustrating a flow of the blood pressure monitoring process according to the present embodiment. Next, the flow of the process of the blood pressure monitor 1A according to the present embodiment will be described with reference to FIG. 6. In addition, the program in accordance with the flowchart is stored in a memory unit 22 in advance, and the control unit 20 executes the process by reading the program from the memory unit 22.

When measuring the blood pressure level, a user wears the cuff 100A by wrapping around the upper arm in advance, and turns on the power source of the blood pressure monitor 1A by operating an operation unit 23 provided in the main body 10A while wearing. Accordingly, electrical power as the power source is supplied to the control unit 20 from the power supply unit 24 to drive the control unit 20. As illustrated in FIG. 6, after receiving such drive, the control unit 20 performs the initialization of the blood pressure monitor 1A (STEP S101).

Next, the control unit 20 waits for the instruction by the user to start measuring, and when the user gives the command to start measuring by operating the operation unit 23, it blocks the air release valve 32 as well as starts driving the pressure pump 31 to gradually increase the cuff pressure of the measuring air bag 130 (STEP S102).

In the process of applying pressure to the measuring air bag 130, the control unit calculates the maximum blood pressure and the minimum blood pressure in a heretofore known procedure (STEP S103). More specifically, the control unit 20 obtains the cuff pressure by an oscillation frequency obtained from the oscillating circuit 36 in the process of applying pressure to the measuring air bag 130, and extracts pulse wave information that is superimposed on the obtained cuff pressure. Then, the control unit 20 calculates the blood pressure level based on the extracted pulse wave information.

After the blood pressure level is calculated in STEP S103, the control unit 20 releases the air completely within the inflate-deflate space 133 of the measuring air bag 130 by opening the air release valve 32 (STEP S104), displays the blood pressure level on the display unit 21 as the measured result as well as stores the blood pressure level to the memory unit 22 (STEP S105).

Subsequently, the control unit 20 ends the operation by waiting for the instruction by the user to turn off the power. In addition, the measurement system described above is based on the so-called pressurization measurement system that detects a pulse at the time of applying pressure to the measuring air bag 130; however, it is also naturally possible to use a decompression measurement system that detects a pulse at the time of reducing pressure of the measuring air bag 130.

In the blood pressure monitor cuff 100A according to the present embodiment described above and the blood pressure monitor 1A equipped therewith, the cuff 100A is given a specified weight distribution by providing the weighted section 160 in the specified position of the cuff 100A. Accordingly, when the user 200 holds the cuff 100A with a hand to wear the cuff 100A, the user 200 is guided to the proper wearing direction of the cuff 100A by feeling the bias of the weight of the cuff 100A. Therefore, the occurrence of the user 200 wearing the cuff 100A in the wrong direction can be prevented beforehand.

Accordingly, by using the configuration described above, unlike the case providing the viewable feature part, the user can be prevented naturally from wearing the cuff in an incorrect manner without paying special attention, and the user is prompted to wear the cuff properly so that the blood pressure measurement can be realized with a high degree of accuracy.

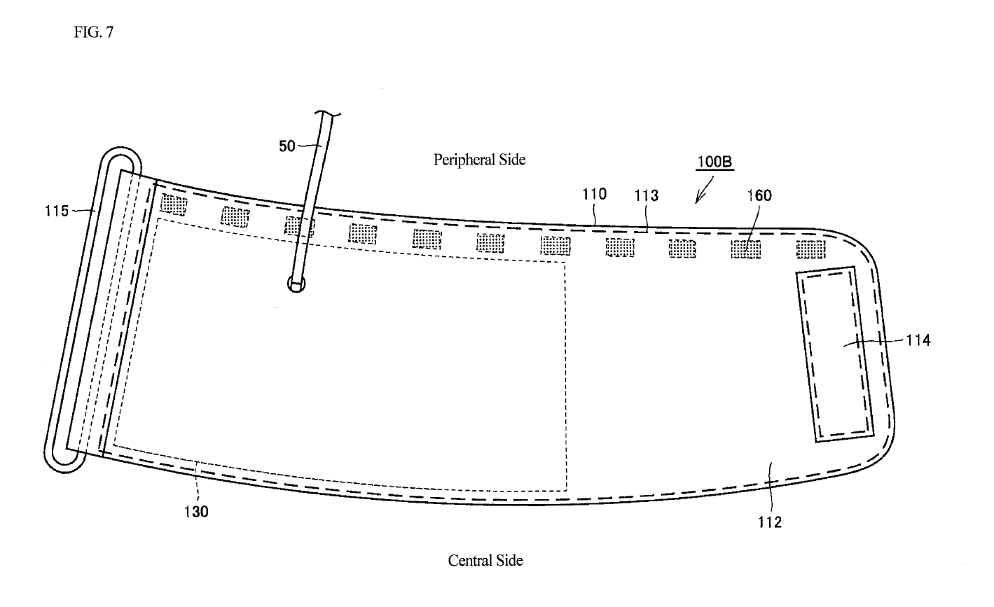

FIG. 7 is a schematic expanded view of the blood pressure monitor cuff that relates to a modified example according to the present embodiment. Next, a blood pressure monitor cuff 100B that relates to the modified example according to the present embodiment will be described with reference to FIG. 7.

In the blood pressure monitor cuff 100B that relates to the present modified example, as illustrated in FIG. 7, a weighted section 160 is provided in the vicinity of the peripheral side end part of an outer cover 110 that is arranged in the peripheral side of the upper arm while wearing in a similar manner to the case of the blood pressure monitor cuff 100A according to the present embodiment described above. Here, in the blood pressure monitor cuff 100B that relates to the present modified example, a plurality of the weighted sections 160 are provided intermittently along the lengthwise direction of the outer cover 110.

In the blood pressure monitor cuff 100B that relates to the present modified example, in addition to a liquid, soft solid matter, aggregated solid particles, or a mixture of these as described above as the weight member contained in the weighted section 160, a non-soft solid matter can be used, and according to one or more embodiments of the present invention, a molded product of a non-pliable metal or the like can be used. This is for preventing deformation of the cuff 100B by arranging the weighted sections splitting into a plurality of members when wearing the cuff 100b wrapped around the upper arm, and by configuring in such a manner, a similar effect to the case of the blood pressure monitor cuff 100A according to the present embodiment described above can be obtained.

### The Second Embodiment

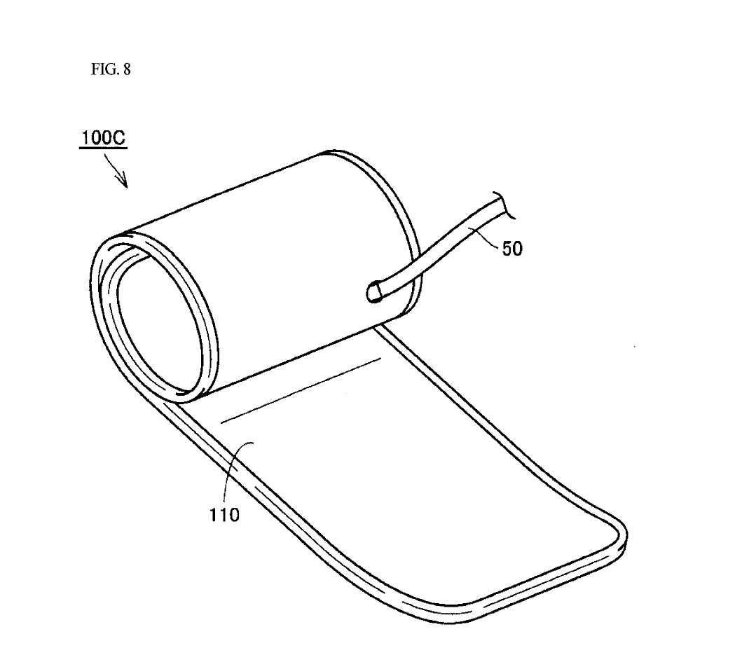

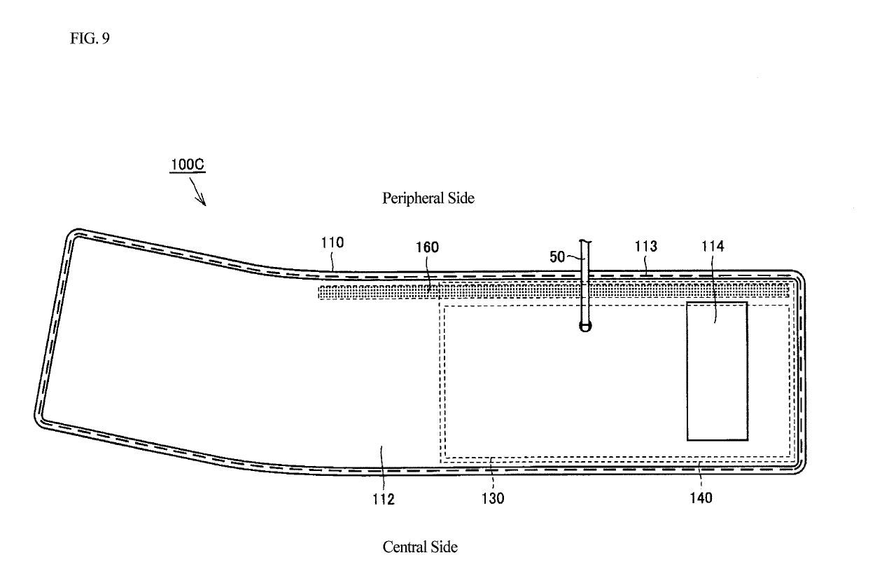

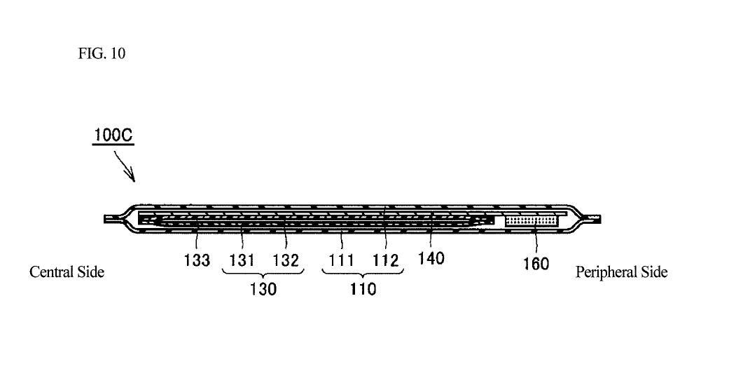

FIG. 8 is a schematic perspective view of a blood pressure monitor cuff according to the second embodiment of the present invention. Further, FIG. 9 is a schematic expanded view of the blood pressure monitor cuff illustrated in FIG. 8, and FIG. 10 is a schematic cross-sectional view. Next, a specific structure of a blood pressure monitor cuff 100C according to the second embodiment of the present invention will be described with reference to FIG. 8 and FIG. 9. In addition, the blood pressure monitor cuff 100C in the present embodiment can be provided to the blood pressure monitor 1A in the first embodiment of the present invention described above with the ability to be replaced with the blood pressure monitor cuff 100A in the first embodiment of the present invention described above.

The blood pressure monitor cuff 100C in the present embodiment, as illustrated in FIG. 8 through FIG. 10, has primarily an outer cover 110, a curler 140, and a measuring air bag 130, and has a configuration of a wrapping shape as well as a circular shape. The cuff 100C is used by wrapping around the upper arm to put on at the time of measuring.

In the blood pressure monitor cuff 100C in the present embodiment, as illustrated in FIG. 9 and FIG. 10, the curler 140 is arranged inside the outer cover 100 and also in the outside position of the measuring air bag 130. The curler 140 is composed of a flexible member that is configured with the ability to elastically deform in a radial direction, and is configured with a curved elastic plate having the circular shape when no external force is applied. The curler 140 is adhesively secured on contact by an intermediary of an adhesive member such as double-sided tape, not illustrated, in the outer circumference surface of the measuring air bag 130, and it is configured so as to follow along the upper arm by maintaining its own circular shape.

In the state where the cuff 100C is expanded as illustrated in FIG. 9, the curler 140 has a substantially rectangular shape in a plan view, and is housed in a housing space of the outer cover 110 so as to cover the measuring air bag 130. In addition, the curler 140 does not reach to one side end where the hook-and-loop fastener 114 of the outer cover 110 is not provided.

The curler 140 is to enable the cuff 100C to be easily put on the upper arm by the user himself or herself, and also to bias the measuring air bag 130 towards the upper arm side when the cuff 100C is worn on the upper arm. Accordingly, in the blood pressure monitor cuff 100C in the present embodiment, the cuff 100C can be easily worn on the upper arm without providing the ring member 115 described above. In addition, the curler 140 is configured with a member composed of preferably polypropylene (PP) resin or the like so as to express elasticity sufficiently.

Here, even in the blood pressure monitor cuff 100C in the present embodiment, as illustrated in FIG. 9 and FIG. 10, a weighted section 160 is provided in the vicinity of the peripheral side end part of the outer cover 110 that is arranged in the peripheral side of the upper arm while wearing. The weighted section 160 is secured on the curler 140 described above by an adhesive or the like so as to be provided by extending along the lengthwise direction of the outer cover 110, and it is preferably configured with a deformable member that is able to deform in any shape.

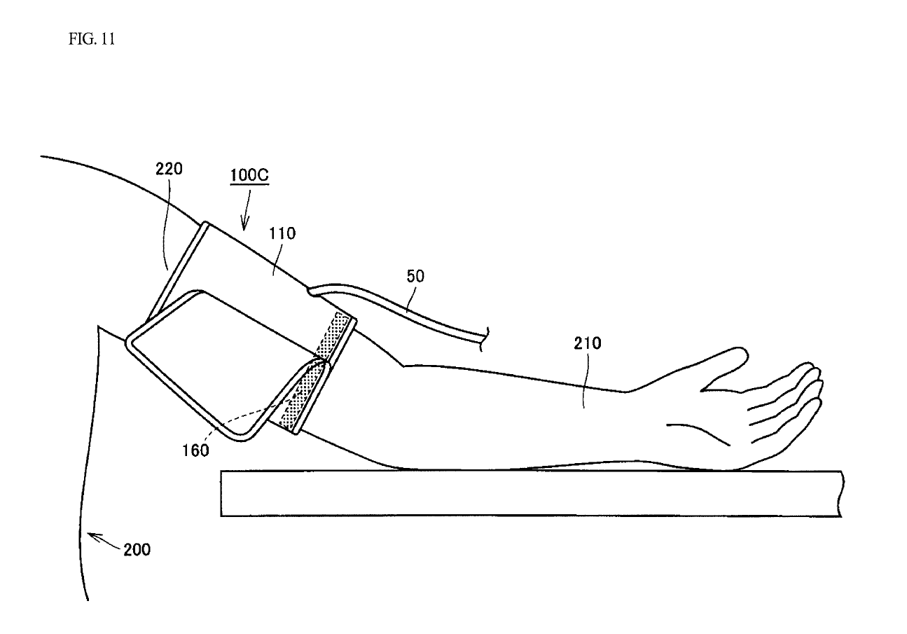

FIG. 11 is a diagram illustrating a state when the blood pressure monitor cuff illustrated in FIG. 8 is worn on the upper arm. Next, a description will be given with reference to FIG. 11 regarding the state when the blood pressure cuff 100C according to the present embodiment is actually worn on the upper arm for measuring the blood pressure.

As illustrated in FIG. 11, the blood pressure monitor cuff 100C according to the present embodiment is used while a user wears it on an upper arm 220 of, for example, the left arm 210. When the cuff is worn, a portion where the curler 140 of the outer cover 110 is not housed is the portion where the curler 140 of the outer cover 110 is housed, and the wrapped cuff 100C is also maintained by securing the hook-and-loop fastener 114 to the portion that is wrapped around the upper arm, and thereby the cuff 100C is worn securely on the upper arm 220.

At such time, the peripheral side end part where the weighted section 160 of the outer cover 110 is provided is arranged in the elbow side position of the upper arm 220, and the central side end part where the weighted section 160 of the outer cover 110 is not provided is arranged in the shoulder side position of the upper arm 220.

In the blood pressure monitor cuff 100C according to the present embodiment described above and the blood pressure monitor 1A equipped therewith, the cuff 100C is given a specified weight distribution by providing the weighted section 160 in the specified position of the cuff 100C. Accordingly, when the user 200 holds the cuff 100C with his/her hand during wearing the cuff 100C, the user 200 is guided to the proper wearing direction of the cuff 100C by feeling the bias of the weight of the cuff 100C. Therefore, the occurrence of the user 200 wearing the cuff 100C in the wrong direction can be prevented beforehand.

Accordingly, by using the configuration described above, unlike the case providing the viewable feature part, the user can be prevented naturally from wearing the cuff in an incorrect manner without paying a special attention, and the user is prompted to wear the cuff properly so that the blood pressure measurement can be realized with a high degree of accuracy.

Note that even when adopting the blood pressure monitor cuff with the curler as described in the present embodiment, it is obvious that the weighted section can be arranged by dividing as described in the modified example according to the embodiment of the present invention above.

### The Third Embodiment

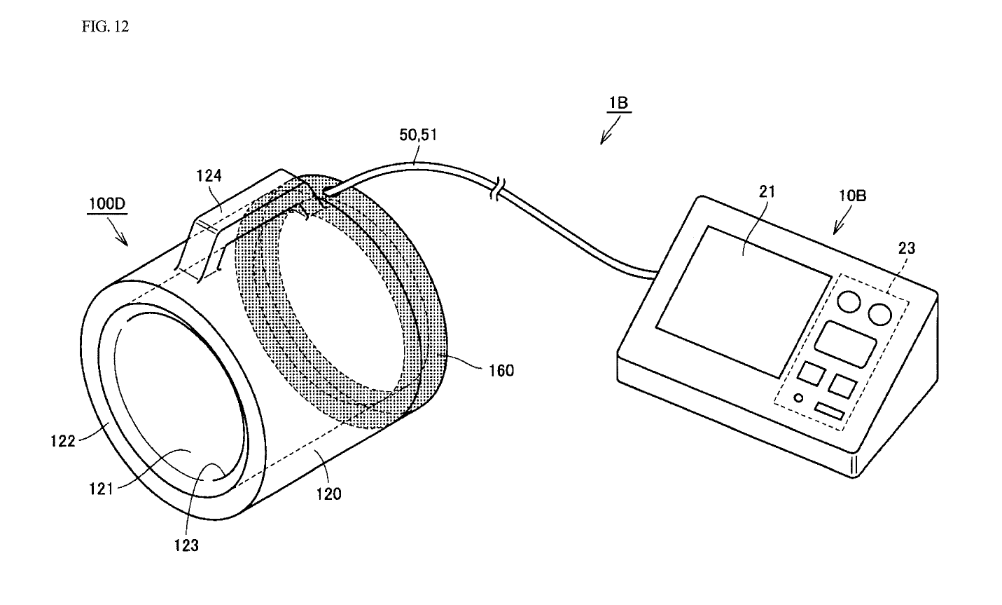

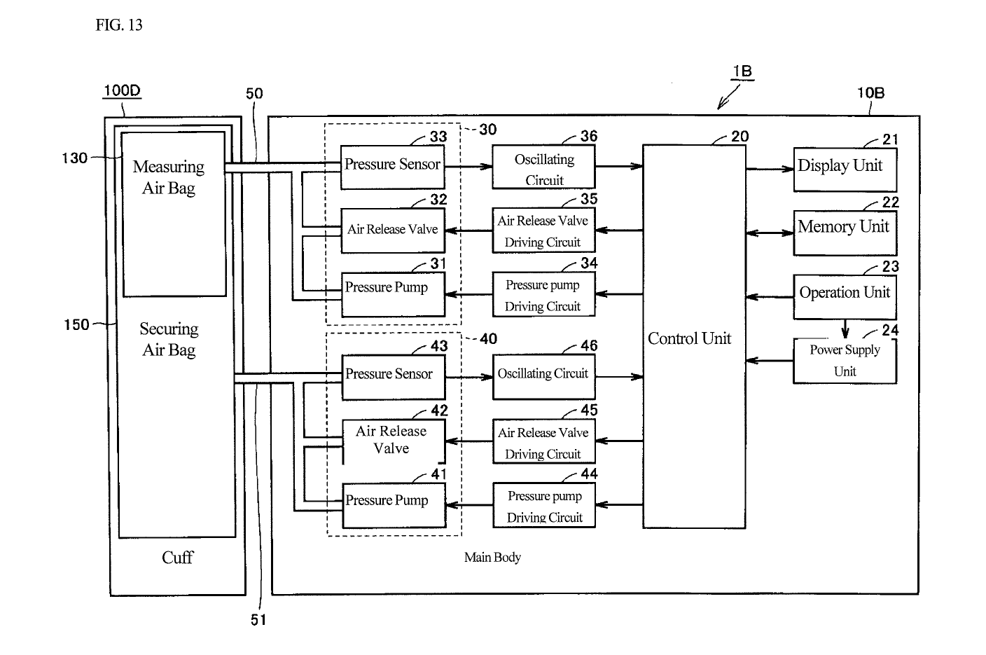

FIG. 12 is a schematic perspective view of a blood pressure monitor cuff according to the third embodiment of the present invention and a blood pressure monitor equipped therewith. Further, FIG. 13 is a diagram illustrating a configuration of a functional block of the blood pressure monitor illustrated in FIG. 12. Next, a description will be given regarding a structure of the exterior view and a configuration of a functional block of a blood pressure monitor 1B according to the third embodiment of the present invention with reference to FIG. 12 and FIG. 13.

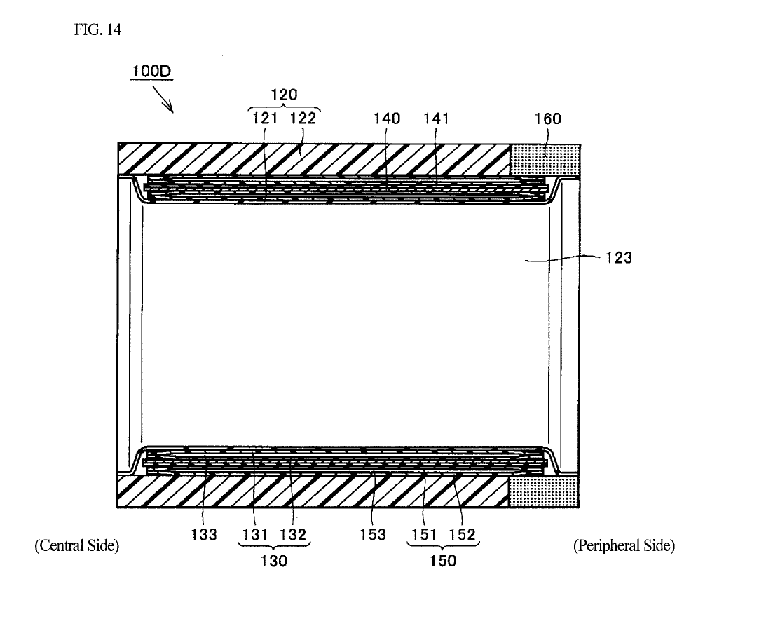

As illustrated in FIG. 12, the blood pressure monitor 1B according to the present embodiment is provided with a main body 10B, a cuff 100D, and air tubes 50, and 51. The external view of the main body 10B is similar to the one in the first embodiment of the present invention described above. The cuff 100D primarily has an outer cover 120 as a tubular-shaped outer body, a measuring air bag 130 and securing air bag 150 (refer to FIG. 13 and FIG. 14), a curler 140 (refer to FIG. 13 and FIG. 14), and has an overall cylindrical shape. The cuff 100D is used by wearing by inserting the upper arm into a hollow section 123 that is formed therein at the time of measuring, and a handle unit 124 is provided in the specified position of the outer circumference surface.

As illustrated in FIG. 13, the main body 10B, in addition to the configuration that is equipped in the main body 10A of the blood pressure monitor 1A in the first embodiment of the present invention described above, further has a pressure pump 41, an air release valve 42, a pressure sensor 43, pressure pump driving circuit 44, an air release valve driving circuit 45, and an oscillating circuit 46. The pressure pump 41, air release valve 42, and the pressure sensor 43 correspond to a securing air system component 40 that is provided in the blood pressure monitor 1B, and particularly the pressure pump 41 and the air release valve 42 correspond to the inflate-deflate mechanism to inflate and deflate the securing air bag 150.

The securing air bag 150 is a fluid bag in order to secure the curler 140 and the measuring air bag 130 to the upper arm by applying compression to the curler 140, and an inflate-deflate space 153 (refer to FIG. 14) is provided as the inner space therein. The securing air bag 150 is connected respectively to the pressure pump 41, air release valve 42 and pressure sensor 43 that are the securing air system component 40 described above through the air tube 51 described above.

The control unit 20, in addition to the function described in the first embodiment of the present invention described above, is further provided with a function to input a control signal for driving the pressure pump 41 and the air release valve 42 to the pressure pump driving circuit 44 and the air release valve driving circuit 45 respectively, and a function to discriminate a secured state on the upper arm of the curler 140 based on the pressure value detected by the pressure sensor 43.

The pressure pump driving circuit 44 controls the operation of the pressure pump 41 based on the control signal input from the control unit 20. The air release valve driving circuit 45 controls the opening-closing operation of the air release valve 42 based on a control signal input from the control unit 20. The pressure pump 41 is for applying pressure to the internal pressure of the securing air bag 150 by supplying air to the inflate-deflate space 153 of the securing air bag 150, and the operation thereof is controlled by the pressure pump driving circuit 44 described above. The air release valve 42 is for maintaining the internal pressure of the securing air bag 150 and depressurizing the internal pressure by releasing the inflate-deflate space 153 of the securing air bag 150 to the outside, and the operation thereof is controlled by the air release valve driving circuit 45 described above. The pressure sensor 43 is a capacitive-type sensor, and the capacitance varies according to the internal pressure of the securing air bag 150. The oscillating circuit 46 generates an oscillation frequency signal in accordance with the capacitance of the pressure sensor 43, and inputs the generated signal to the control unit 20.

FIG. 14 is a schematic cross-sectional view of a blood pressure monitor cuff illustrated in FIG. 12. Next, a specific structure of the blood pressure monitor cuff 100D according to the present embodiment will be described with reference to FIG. 14 and FIG. 12 described above.

The blood pressure monitor cuff 100D in the present embodiment, as illustrated in FIG. 12 and FIG. 14, has a measuring air bag connected to an air tube 50 and a securing air bag 150 connected to an air tube 51, a curler 140, and an outer cover 120 as a tubular-shaped outer body that contains these measuring air bag 130, securing air bag 150, and the curler 140. The measuring air bag 130, securing air bag 150, and the curler 140 all have a substantially rectangular shape in a plan view when they are expanded, and these are overlapped and made in a circular shape and housed inside the outer cover 120 as the tubular-shaped outer body.

As illustrated in FIG. 14, according to one or more embodiments of the present invention, the securing air bag 150 is composed of a bag-like member formed by using a resin sheet. More specifically, the securing air bag 150 has an inner sheet member 151 that is placed in the upper arm side when the cuff 100D is worn on the upper arm, and an outer sheet member 152 that is placed in the outer side of the inner sheet member 151 when the cuff 100D is worn on the upper arm, and the inflate-deflate space 153 described above is provided therein. The securing air bag 150 is formed in a bag-like shape by laying the inner sheet member 151 and the outer sheet member 152 on top of each other and welding the peripheral edges thereof.

For the resin sheet that configures the securing air bag 150, any material may be used as long as it has elasticity and there is no air leakage from the inflate-deflate space 153 after the welding. From such a standpoint, according to one or more embodiments of the present invention, a material made of; such as, an ethylene-vinyl acetate (EVA) copolymer resin, flexible polyvinyl chloride (PVC) resin, polyurethane (PU) resin, polyamide (PA) resin, and the like can be used.

Further, the outer cover 120, as illustrated in FIG. 12 and FIG. 14, has a cover member 121 placed in a hollow section 123 side where the upper arm is inserted, and a shell 122 placed in the outside of the cover member 121, and a housing space to store the measuring air bag 130 or the like described above is provided therein. The outer cover 120 is configured such that the periphery of the cover member 121 is fixed to the periphery of the inner circumference surface of the shell 122 having a tubular shape.

The shell 122 is configured of a hard member with inflexibility, and according to one or more embodiments of the present invention, the shell 122 is configured with a member composed of an acrylonitrile-butadiene-styrene (ABS) copolymer resin and the like. On the other hand, the outer cover 121 has flexibility, and according to one or more embodiments of the present invention, the outer cover 121 is composed of a cloth made of a synthetic fiber such as a polyamide (PA) resin, polyester resin, and the like. As described above, the outer cover 120 is configured so as to have inflexibility at least in the outer circumference part. In addition, according to one or more embodiments of the present invention, the cover member 121 is configured with a member having excellent elasticity.

In addition, the shape and material of the measuring air bag 130 is fundamentally similar to the one in the first embodiment of the present invention described above, and the shape and material of the curler 140 is similar to the one in the second embodiment of the present invention described above. However, in the cuff 100D in the present embodiment, a sheet-formed cloth 141 as a low friction member is arranged between the curler 140 and the securing air bag 150 in order to enhance sliding between the curler 140 and the securing air bag 150.

In the blood pressure monitor cuff 100D in the present embodiment, as illustrated in FIG. 12 and FIG. 14, a weighted section 160 is provided in the vicinity of an axial direction end part of the outer cover 120 that is arranged in the peripheral side of the upper arm while wearing. The weighted section 160 is provided by extending along the circumferential direction of the outer cover 120, and it has a circular shape.

The weighted section 160 contains a weight member that has a greater specific gravity than other members that configure the cuff 100D. For the weight member, a liquid, a soft solid matter, a non-soft solid matter, aggregated solid particles, or a mixture of these is used, and according to one or more embodiments of the present invention, a liquid such as water, a molded product made of a soft metal or non-soft metal, aggregated solid particles (powder) such as sand, metal, or the like, a mixture of these, or the like is used. In addition, according to one or more embodiments of the present invention, when a liquid or aggregated solid particles is used as the weight member, these weight members are housed in a sealed body.

In addition, the installation method for the outer cover 120 of the weighted section 160 is not particularly restricted, and as illustrated in FIG. 14, a portion of the shell 122 may be configured in the weighted section 160, and the weighted section 160 may be arranged on the inner circumference surface or on the outer circumference surface, or the weighted section 160 may be embedded inside the shell 122. Further, the weighted section 160 may also be attached to the cover member 121 or the curler 140.

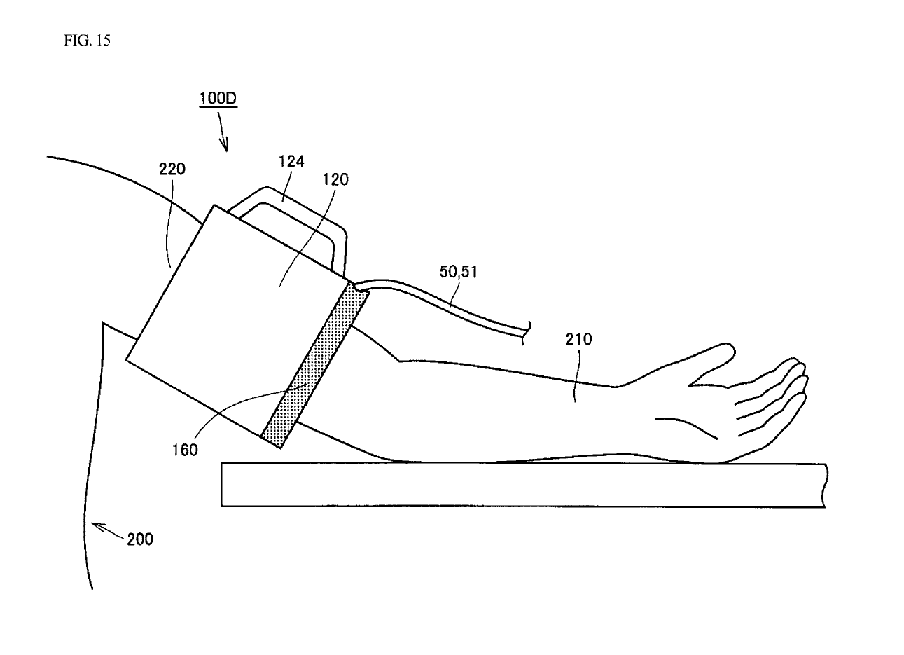

FIG. 15 is a diagram illustrating a state when the blood pressure monitor cuff illustrated in FIG. 12 is worn on the upper arm. Next, a description will be given with reference to FIG. 15 regarding the state when the blood pressure cuff 100D according to the present embodiment is actually worn on the upper arm for measuring the blood pressure.

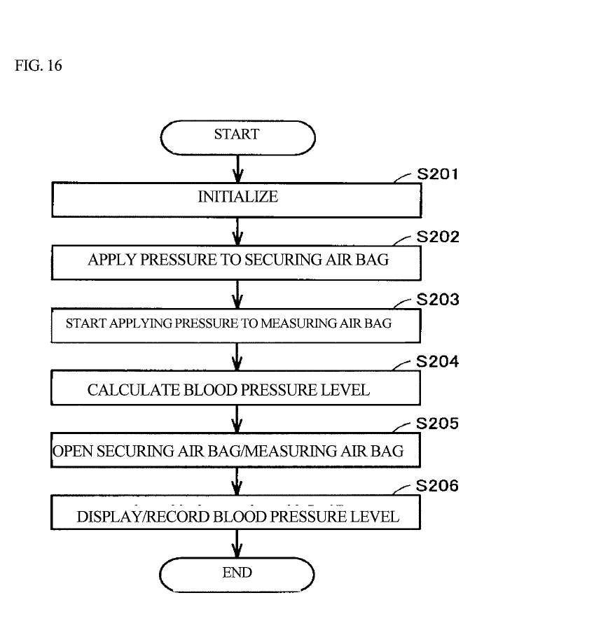

As illustrated in FIG. 15, the blood pressure monitor cuff 100D according to the present embodiment is used while a user wears it on an upper arm 220, for example, of the left arm 210. When wearing, the right hand grips the handle unit 124 provided on the cuff 100D, and in such condition, the upper arm 220 of the left arm 210 is inserted in the hollow section 123 of the cuff 100D having a cylindrical shape. Then, the cuff 100D is secured to the upper arm 220 by operating the operation unit 23 provided in the main body 10B to push down the measuring button to execute the processing procedure illustrated in FIG. 16 as will be described later, and afterward the blood pressure measurement is performed.

Here, when the cuff 100D is placed on the upper arm 220, the axial direction end part where the weighted section 160 of the outer cover 120 is provided is arranged on the elbow side position (in other words, the peripheral side) of the upper arm 220, and the axial direction end part where the weighted section 160 of the outer cover 120 is not provided is arranged in the shoulder side position (in other words, the central side) of the upper arm 220.

FIG. 16 is a flowchart illustrating a flow of the blood pressure monitor process according to the present embodiment. Next, the flow of the process of the blood pressure monitor 1B according to the present embodiment will be described with reference to FIG. 16. In addition, the program in accordance with the flowchart is stored in advance in a memory unit 22, and the control unit 20 executes the process by reading the program from the memory unit 22.

When measuring the blood pressure level, a user inserts the upper arm into the cuff 100D in advance, and turns on the power source of the blood pressure monitor 1B by operating the operation unit 23 provided in the main body 10B while wearing. Accordingly, electrical power is supplied as the power source to the control unit 20 from the power supply unit 24 to drive the control unit 20. As illustrated in FIG. 16, after such driving, the control unit 20 performs the initialization of the blood pressure monitor 1B (STEP S201).

Next, the control unit 20 waits for the instruction by the user to start measuring, and when the user gives the command to start measuring by operating the operation unit 23, it blocks the air release valve 42 as well as starts driving the pressure pump 41 to increase the internal pressure of the securing air bag 150 until reaching a specified value (STEP S202).

Next, the control unit 20 blocks the air release valve 32 at the time when the internal pressure of the securing air bag 150 reaches the specified level, and at the same time, increases gradually the cuff pressure of the measuring air bag 130 (STEP S203).

In the process of applying pressure to the measuring air bag 130, the control unit calculates the maximum blood pressure and the minimum blood pressure in a heretofore known procedure (STEP S204). In addition, the specific operations are similar to those in the first embodiment of the present invention described above.

After the blood pressure level is calculated in STEP S204, the control unit 20 releases the air completely within the inflate-deflate space 153 of the securing air bag 150 by opening the air release valve 42, and at the same time, releases the air within the inflate-deflate space 133 of the measuring air bag 130 by opening the air release valve 32 (STEP S205), and displays the blood pressure level in the display unit 21 as the measured result as well as stores the blood pressure level to the memory unit 22 (STEP S206).

Subsequently, the control unit 20 ends the operation by waiting for the instruction by the user to turn off the power. In addition, the measurement system described above is based on the so-called pressurization measurement system that detects a pulse at the time of applying pressure to the measuring air bag 130; however, it is also obvious that a decompression measurement system that detects a pulse at the time of reducing pressure of the measuring air bag 130 can also be used.

In the blood pressure monitor cuff 100D according to the present embodiment described above and the blood pressure monitor 1B equipped therewith, the cuff 100D is given a specified weight distribution by providing the weighted section 160 in a specified position of the cuff 100D. Accordingly, when the user 200 holds the cuff 100D with his/her hand while wearing the cuff 100D, the user 200 is guided to the proper wearing direction of the cuff 100D by feeling the bias of the weight of the cuff 100D. Therefore, the occurrence of the user 200 wearing the cuff 100D in the wrong direction can be prevented beforehand.

Accordingly, by using the configuration described above, unlike the case providing the viewable feature part, the user can be prevented naturally from wearing the cuff in an incorrect manner without paying special attention, and the user is prompted to wear the cuff properly so that the blood pressure measurement can be realized with a high degree of accuracy.

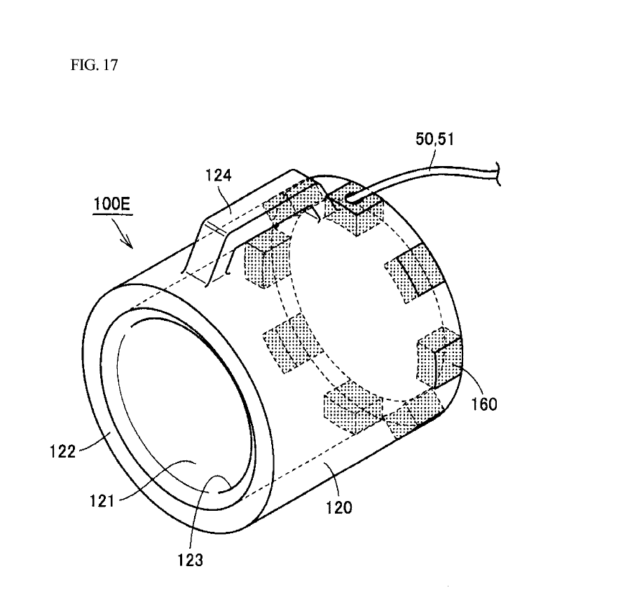

FIG. 17 is a schematic perspective view of a blood pressure monitor cuff that relates to a modified example according to the present embodiment. Next, a blood pressure monitor cuff 100E that relates to a modified example according to the present embodiment will be described with reference to FIG. 17.

In the blood pressure monitor cuff 100E that relates to the present modified example, as illustrated in FIG. 17, a weighted section 160 is provided in the vicinity of the axial direction side end part of an outer cover 120 that is arranged on the peripheral side of the upper arm while wearing that is similar to the case of the blood pressure monitor a cuff 100D according to the present embodiment described above. Here, in the blood pressure monitor cuff 100E that relates to the present modified example, a plurality of the weighted sections 160 are provided intermittently along the circumferential direction of the outer cover 120. With the configuration in such a manner, this can obtain similar effects as the blood pressure monitor cuff 100D in the present embodiment described above.

### The Fourth Embodiment

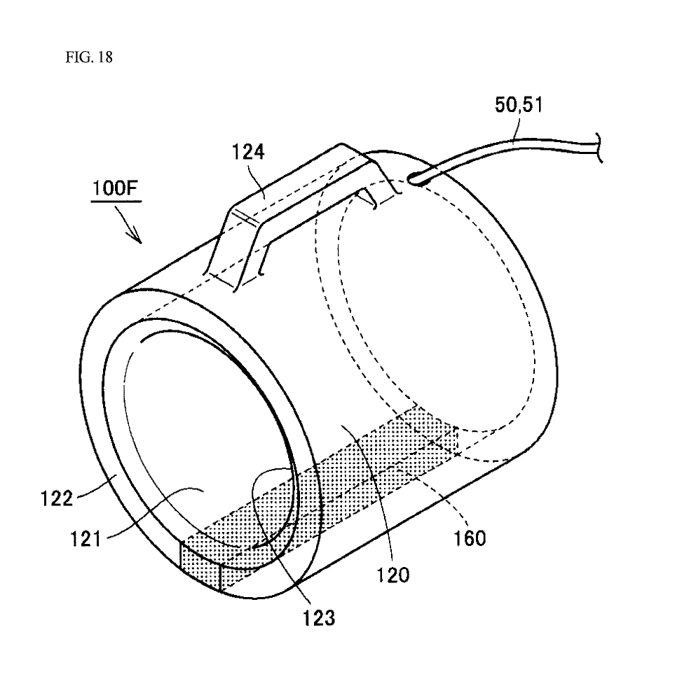

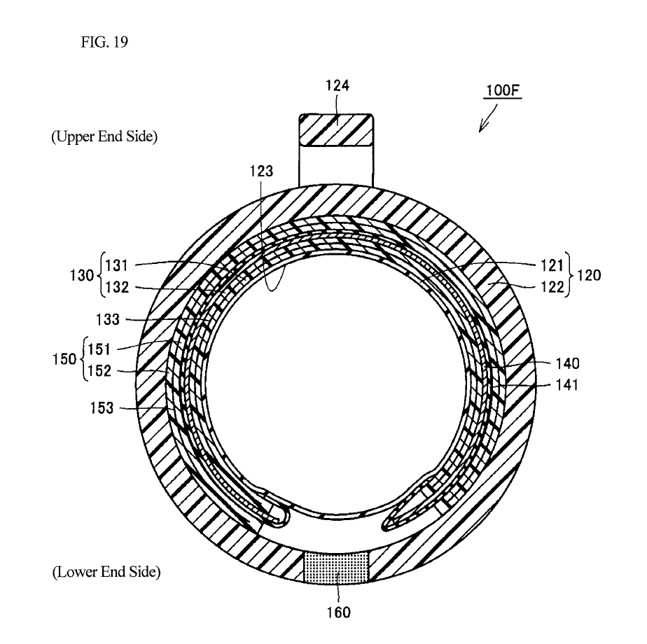

FIG. 18 is a schematic perspective view of a blood pressure monitor cuff according to the fourth embodiment of the present invention. Further, FIG. 19 is a schematic cross-sectional view of the blood pressure monitor cuff illustrated in FIG. 18. Next, a specific structure of a blood pressure monitor cuff 100F according to the fourth embodiment of the present invention will be described with reference to FIG. 18 and FIG. 19. In addition, the blood pressure monitor cuff 100F in the present embodiment can be provided to the blood pressure monitor 1B in the third embodiment of the present invention described above with the ability to be replaced with the blood pressure monitor cuff 100D in the third embodiment of the present invention described above.

In the blood pressure monitor cuff 100F in the present embodiment, as illustrated in FIG. 18 and FIG. 19, a weighted section 160 is provided in the vicinity of the circumferential direction lower end part positioned on the most vertical lower part within the lower side part of the outer cover 120 that is arranged vertically downward when wearing if the outer cover 120 is split into two in a plane including the axis line. The weighted section 160 is provided by extending along the axial direction of the outer cover 120. Here, because a handle unit 124 is provided in the vicinity of the circumferential upper end part that is placed in the most vertical upper part within the upper side part of the outer cover 120 that is arranged vertically upward when wearing, the weighted section 160 and the handle unit 124 are provided in the position directly opposed on the top and bottom having a hollow section therebetween.

The weighted section 160 contains a weight member that has a greater specific gravity than other members that configure the cuff 100F. For the weight member, a liquid, a soft solid matter, a non-soft solid matter, aggregated solid particles, or a mixture of these is used, and according to one or more embodiments of the present invention, a liquid such as water, a molded product made of a soft metal or non-soft metal, aggregated solid particles (powder) such as sand, metal, or the like, a mixture of these, or the like is used. In addition, when a liquid or aggregated solid particles is used as the weight member, according to one or more embodiments of the present invention, these weight members are sealed in a sealed body.

In addition, the installation method for the outer cover 120 of the weighted section 160 is not particular restricted, and as illustrated in FIG. 19, a portion of the shell 122 may be configured in the weighted section 160, and the weighted section 160 may be arranged on the inner circumference surface or on the outer circumference surface, or the weighted section 160 may be embedded inside the shell 122.

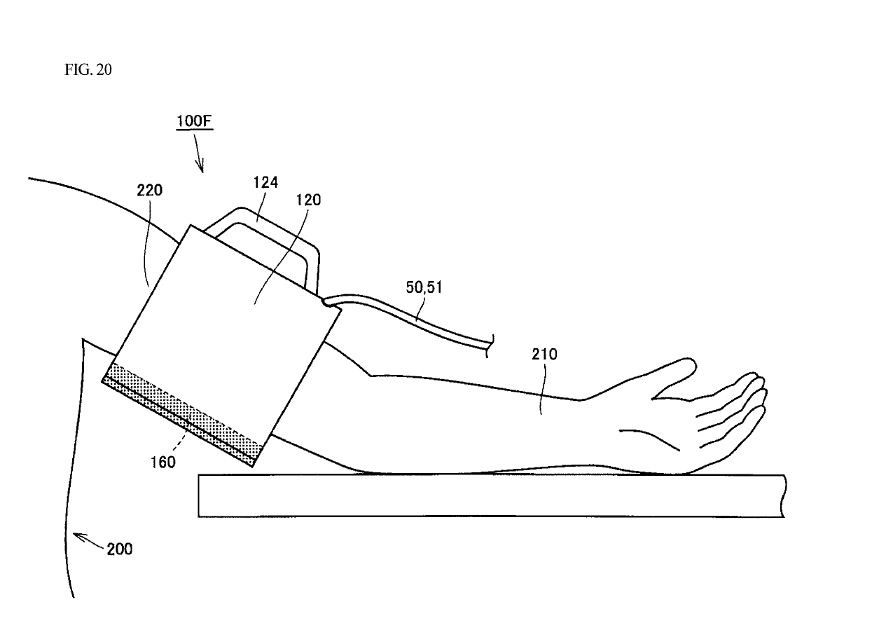

FIG. 20 is a diagram illustrating a state when the blood pressure monitor cuff illustrated in FIG. 18 is worn on the upper arm. Next, a description will be given with reference to FIG. 20 regarding the state when the blood pressure cuff 100F according to the present embodiment is actually worn on the upper arm for measuring the blood pressure.

As illustrated in FIG. 20, the blood pressure monitor cuff 100F according to the present embodiment is used while a user wears it on an upper arm 220 of; for example, the left arm 210. When wearing, the right hand grips the handle unit 124 provided on the cuff 100F, and in such condition, the upper arm 220 of the left arm 210 is inserted in the hollow section 123 of the cuff 100F having a cylindrical shape. Then, the cuff 100F is secured to the upper arm 220 by operating the operation unit 23 provided in the main body 10B to push down the measuring button to execute the processing procedure illustrated in FIG. 16 described above, and thereafter the blood pressure measurement is performed.

Here, when the cuff 100F is placed to the upper arm 220, the circumferential direction lower part where the weighted section 160 of the outer cover 120 is provided is arranged in the vertical downward position of the upper arm 220, and the circumferential upper end part where the handle unit 124 of the outer cover 120 is provided is arranged in the vertical upward position of the upper arm 220.

In the blood pressure monitor cuff 100F according to the present embodiment described above and the blood pressure monitor 1B equipped therewith, the cuff 100F is given a specified weight distribution by providing the weighted section 160 in the specified position of the cuff 100F. Accordingly, when the user 200 holds the cuff 100F with a hand to wear the cuff 100F, the user 200 is guided to wear in the proper arrangement in the top-bottom direction of the cuff 100F (in other words, in a state where positioning of the wearing position in the circumferential direction of the cuff 100F is performed) by feeling the bias of the weight of the cuff 100F. Therefore, the occurrence of the user 200 wearing the cuff 100F in a wrong direction can be prevented beforehand.

Accordingly, by using the configuration described above, unlike the case providing the viewable feature part, the user can be prevented naturally from wearing the cuff in an incorrect manner without paying special attention, and the user is prompted to wear the cuff properly so that the blood pressure measurement can be realized with a high degree of accuracy.

In addition, in the blood pressure monitor cuff 100F in the present embodiment, the handle unit 124 is provided in the vicinity of the circumferential direction upper end part of the outer cover 120 that is arranged in the vertically upward position while wearing; however, the placement position of the handle unit 124 is not limited to the circumferential direction upper end part of the outer cover 120, and it may be provided in any position as long as it is in the position included in the upper side part of the outer cover 120 (in other words, any position of the upper half).

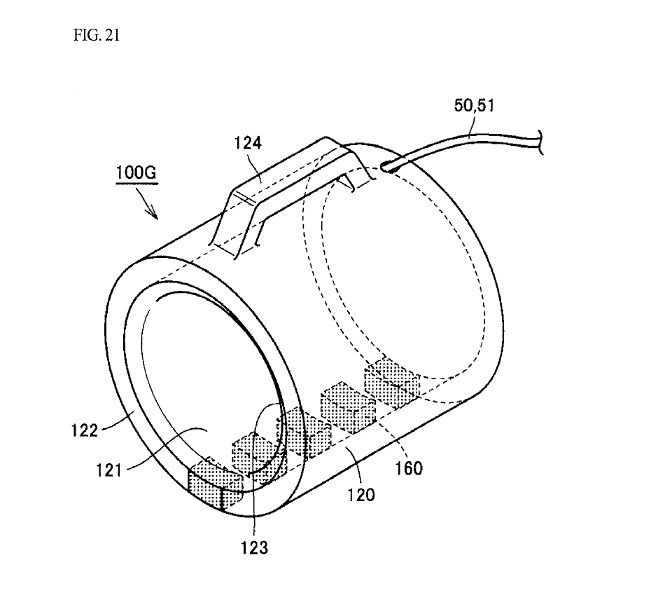

FIG. 21 is a schematic perspective view of a blood pressure monitor cuff that relates a first modified example according to the present embodiment. Next, a blood pressure monitor cuff 100G that relates to the first modified example according to the present embodiment will be described with reference to FIG. 21.

As is the case with the blood pressure monitor cuff 100F in the present embodiment described above, in the blood pressure monitor cuff 100G that relates to the present first modified example, as illustrated in FIG. 21, a weighted section 160 is provided in the vicinity of the circumferential direction lower end part positioned in the most vertically lower part within the lower side part of the outer cover 120 that is arranged vertically downward when wearing if the outer cover 120 is split into two in a plane including the axis line. Here, in the blood pressure monitor cuff 100G that relates to the present first modified example, a plurality of the weighted sections 160 are provided intermittently along the axial direction of the outer cover 120. With the configuration in such a manner, this can obtain similar effects as the blood pressure monitor cuff 100F in the present embodiment described above.

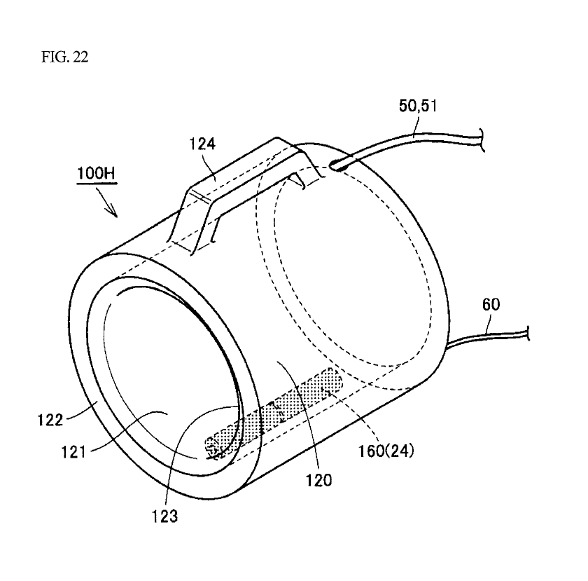

FIG. 22 is a schematic perspective view of a blood pressure monitor cuff that relates to a second modified example according to the present embodiment. Next, a blood pressure monitor cuff 100H that relates to the second modified example according to the present embodiment will be described with reference to FIG. 22.

As is the case with the blood pressure monitor cuff 100F in the present embodiment described above, in the blood pressure monitor cuff 100H that relates to the present second modified example, as illustrated in FIG. 22, a weighted section 160 is provided in the vicinity of the circumferential direction lower end part positioned in the most vertically lower part within the lower side part of the outer cover 120 that is arranged vertically downward when wearing if the outer cover 120 is split into two in a plane including the axis line. Here, in the blood pressure monitor cuff 100H that relates to the present second modified example, the weighted section 160 is configured by a battery as the power supply unit 24. In other words, because a battery normally can be a weight member with a greater specific gravity than other members that configure the cuff 100H, and therefore, by using the battery as the weighted section 160, there is no need to configure the weighted section by providing a weight member separately, and this leads to an advantage from the standpoint of space saving. As a result, with the configuration even in such a manner, this can obtain similar effects as the blood pressure monitor cuff 100F in the present embodiment described above.

In addition, when a battery that is the electrical supply unit 24 is provided in the cuff 100H such as the present second modified example, the cuff 100H and the main body 10B are connected with a connecting cable 60 as illustrated in the drawing due to the necessity of transmitting electrical power supplied from the battery. However, when this is integrated with the cuff 100H without providing the main body 10B, air tubes 50 and 51 are no longer unnecessary but the connection with the connecting cable 60 is also naturally no longer needed.

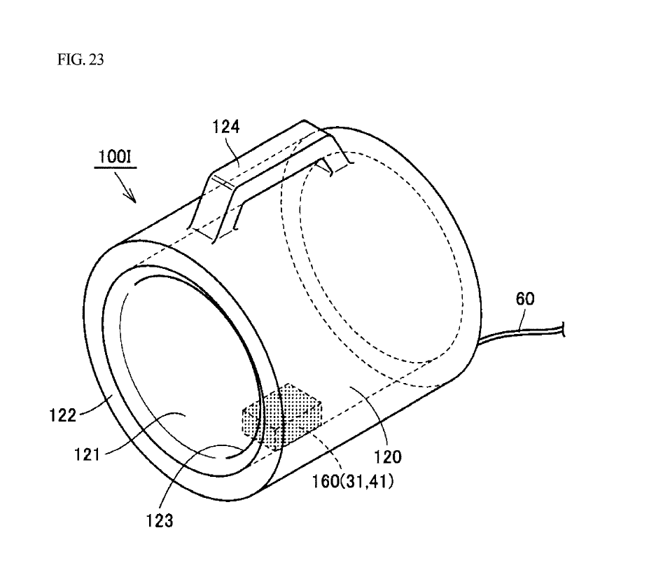

FIG. 23 is a schematic perspective view of a blood pressure monitor cuff that relates a third modified example according to the present embodiment Next, a blood pressure monitor cuff 100I that relates to the third modified example according to the present embodiment will be described with reference to FIG. 23.

As is the case with the blood pressure monitor cuff 100F in the present embodiment described above, in the blood pressure monitor cuff 100I that relates to the present third modified example, as illustrated in FIG. 23, a weighted section 160 is provided in the vicinity of the circumferential direction lower end part positioned in the most vertically lower part within the lower side part of the outer cover 120 that is arranged vertically downward when wearing if the outer cover 120 is split into two in a plane including the axis line. Here, in the blood pressure monitor cuff 100I that relates to the present third modified example, the weighted section 160 is configured by pressure pumps 31 and 41. In other words, because the pressure pumps 31 and 41 normally can be a weight member with a greater specific gravity than other members that configure the cuff 100I, by using the pressure pumps 31 and 41 as the weighted sections 160, there is no need to configure the weighted section by providing a separate weight member, and this leads to an advantage from the standpoint of space saving. As a result, with the configuration even in such a manner, this can obtain similar effects as the blood pressure monitor cuff 100F in the present embodiment described above.

In addition, when the pressure pumps 31 and 41 are provided in the cuff 100I such as according to the present third modified example, the air tubes 50 and 51 are unnecessary to be provided by further providing the air release valves 32 and 42 and the pressure sensors 33 and 43 as illustrated in the drawing. However, a control signal is necessary to be transmitted or the like to the cuff 100I from the main body 10B to drive the pressure pumps 31 and 41, and therefore, the cuff 100I and the main body 10B are required to be connected by the connecting cable 60 as illustrated in the drawing. However, if this is integrated with the cuff 100I without providing the main body 10B, the connection by the connecting cable 60 is naturally no longer needed.

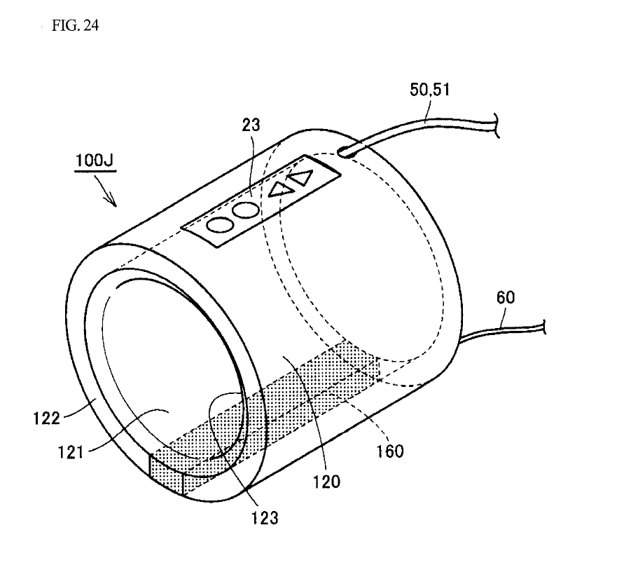

FIG. 24 is a schematic perspective view of a blood pressure monitor cuff that relates to a fourth modified example according to the present embodiment. Next, a blood pressure monitor cuff 100J that relates to the fourth modified example according to the present embodiment will be described with reference to FIG. 24.

As is the case with the blood pressure monitor cuff 100F in the present embodiment described above, in the blood pressure monitor cuff 100J that relates to the present fourth modified example, as illustrated in FIG. 24, a weighted section 160 is provided in the vicinity of the circumferential direction lower end part positioned in the most vertically lower part within the lower side part of the outer cover 120 that is arranged vertically downward when wearing if the outer cover 120 is split into two in a plane including the axis line. Here, in the blood pressure monitor cuff 100J that relates to the present fourth modified example, an operation unit 23 is provided, instead of the handle unit 124, in the vicinity of the circumferential direction upper end part positioned most vertically upward on the upper side part of the outer cover 120 that is arranged vertically upward while wearing. With the configuration even in such a manner, this can obtain similar effects as the blood pressure monitor cuff 100F in the present embodiment described above.

In addition, when the operation unit 23 is provided in the cuff 100J such as the present fourth modified example, the cuff 100J and the main body 10B are required to be connected by a connecting cable 60 as illustrated in the drawing because signals according to the operation of the operation unit 23 are required to be transmitted to the main body 10B from the cuff 100J. However, if this is integrated with the cuff 100J without providing the main body 10B, the connection by the connecting cable 60 is naturally no longer needed.

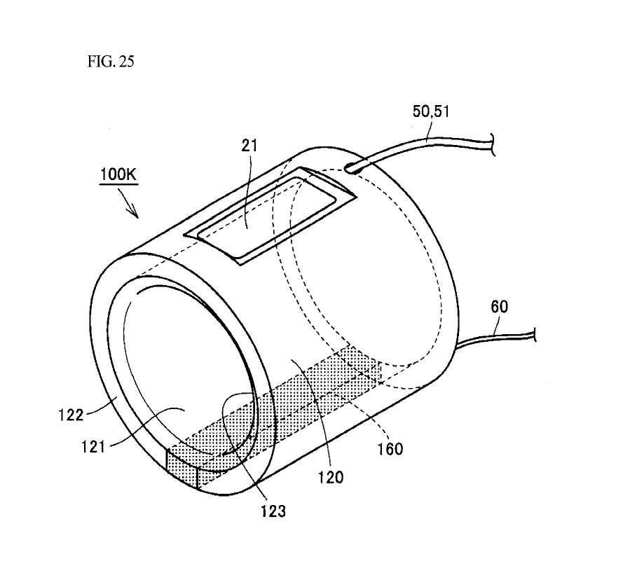

FIG. 25 is a schematic perspective view of a blood pressure monitor cuff that relates to a fifth modified example according to the present embodiment. Next, a blood pressure monitor cuff 100K that relates to the fifth modified example according to the present embodiment will be described with reference to FIG. 25.

As is the case with the blood pressure monitor cuff 100F in the present embodiment described above, in the blood pressure monitor cuff 100K that relates to the present fifth modified example, as illustrated in FIG. 25, a weighted section 160 is provided in the vicinity of the circumferential direction lower end part positioned in the most vertically lower part in the lower side part of the outer cover 120 that is arranged vertically downward when wearing if the outer cover 120 is split into two in a plane including the axis line. Here, in the blood pressure monitor cuff 100K that relates to the present fifth modified example, a display unit 21 is provided, instead of the handle unit 124, in the vicinity of the circumferential direction upper end part positioned most vertically upward in the upper side part of the outer cover 120 that is arranged vertically upward when wearing. With the configuration even in such a manner, this can obtain similar effects as the blood pressure monitor cuff 100F in the present embodiment described above.

In addition, when the display unit 21 is provided in the cuff 100K such as in the present fifth modified example, the cuff 100K and the main body 10B are required to be connected by a connecting cable 60 as illustrated in the drawing because control signals in order to drive the display unit 21 are required to be transmitted to the cuff 100K from the main body 10B. However, if this is integrated with the cuff 100K without providing the main body 10B, the connection by the connecting cable 60 is naturally no longer needed.

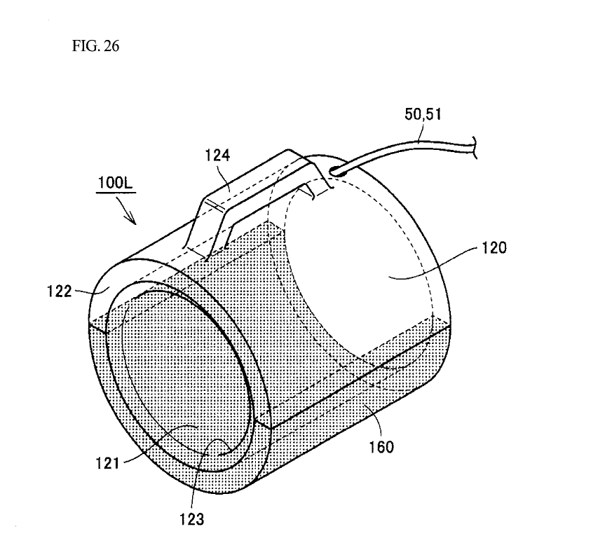

FIG. 26 is a schematic perspective view of a blood pressure monitor cuff that relates to a sixth modified example according to the present embodiment. Next, a blood pressure monitor cuff 100L that relates to the sixth modified example according to the present embodiment will be described with reference to FIG. 26.

In the blood pressure monitor cuff 100L that relates to the present sixth modified example, as illustrated in FIG. 26, a weighted section 160 is provided in the vicinity of the lower side part of the outer cover 120 that is arranged vertically downward when wearing if the outer cover 120 is split into two in a plane including the axis line. Here, in the blood pressure monitor cuff 100L that relates to the present sixth modified example, the weighted sections 160 are provided in all positions of the lower side part of the outer cover 120. With the configuration in such a manner, this can obtain similar effects as the blood pressure monitor cuff 100F in the present embodiment described above.

### The Fifth Embodiment

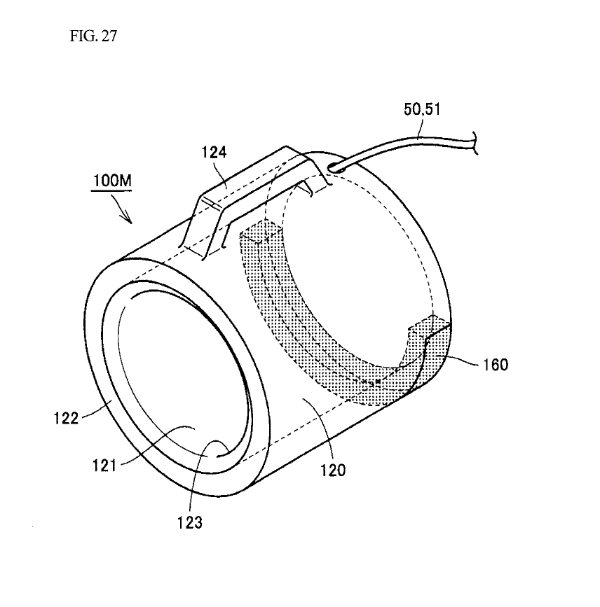

FIG. 27 is a schematic perspective view of a blood pressure monitor cuff according to the second embodiment of the present invention. Next, a specific structure of a blood pressure monitor cuff 100M in the fifth embodiment of the present invention will be described with reference to FIG. 27. In addition, the blood pressure monitor cuff 100M in the present embodiment can be provided to the blood pressure monitor 1B in the third embodiment of the present invention described above with the ability to be replaced with the blood pressure monitor cuff 100D in the third embodiment of the present invention described above.

In the blood pressure monitor cuff 100M in the present embodiment, as illustrated in FIG. 27, a weighted section 160 is provided in the vicinity of the axial direction end part that is a lower side part of the outer cover 120 that is arranged vertically downward and is also required to be arranged on a peripheral side of the upper arm when wearing if the outer cover 120 is split into two in a plane including the axis line. The weighted section 160 is provided by extending along the circumferential direction of the outer cover 120, and it has a half-circular shape.

The weighted section 160 contains a weight member that has a greater specific gravity than other members that configure the cuff 100M. For the weight member, a liquid, a soft solid matter, a non-soft solid matter, aggregated solid particles, or a mixture of these is used, and according to one or more embodiments of the present invention, a liquid such as water, a molded product made of a soft metal or non-soft metal, aggregated solid particles (powder) such as sand, metal, or the like, a mixture of these, or the like is used. In addition, when a liquid or aggregated solid particles is used as the weight member according to one or more embodiments of the present invention, these weight members are sealed in a sealed body.

In addition, the installation method for the outer cover 120 of the weighted section 160 is not particularly restricted, and a portion of the shell 122 may be configured in the weighted section 160, and the weighted section 160 may be arranged on the inner circumference surface or on the outer circumference surface, or the weighted section 160 may be embedded inside the shell 122.

In the blood pressure monitor cuff 100M according to the present embodiment described above and the blood pressure monitor 1B equipped therewith, the cuff 100M is given a specified weight distribution by providing the weighted section 160 in the specified position of the cuff 100M. Accordingly, when the user 200 holds the cuff 100M with a hand to wear the cuff 100M, the user 200 is guided to wear it in the proper arrangement state in the top and bottom direction as well as the front and back direction of the cuff 100M by feeling the bias of the weight of the cuff 100F. Therefore, the occurrence of the user 200 wearing the cuff 100M in the wrong direction can be prevented beforehand.

Accordingly, by using the configuration described above, unlike the case providing the viewable feature part, the user can be prevented naturally from wearing the cuff in an incorrect manner without paying special attention, and the user is prompted to wear the cuff properly so that the blood pressure measurement can be realized with a high degree of accuracy.

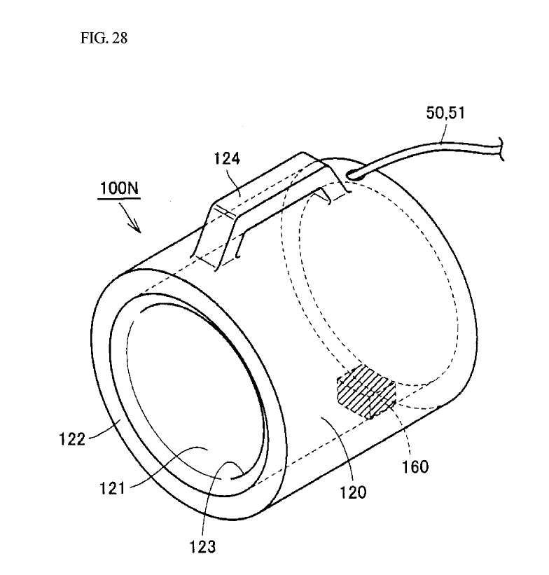

FIG. 28 is a schematic perspective view of a blood pressure monitor cuff that relates to a modified example according to the present embodiment. Next, a blood pressure monitor cuff 100N that relates to the modified example according to the present embodiment will be described with reference to FIG. 28.

As is the case with the blood pressure monitor cuff 100M in the present embodiment described above, in the blood pressure monitor cuff 100N that relates to the present modified example, as illustrated in FIG. 28, a weighted section 160 is provided in the vicinity of the axial direction end part that is a lower side part of the outer cover 120 that is arranged vertically downward and is also required to be arranged on a peripheral side of the upper arm when wearing if the outer cover 120 is split into two in a plane including the axis line. However, the weighted section 160 is not extended along the circumferential direction of the outer cover 120, but is arranged locally. In even such a configuration, this can also obtain similar effects as the blood pressure monitor cuff 100M in the present embodiment described above by giving sufficient weight to the weighted section 160.

These characteristic configurations illustrated in the first embodiment through the fifth embodiment and the modified examples of the present invention described above are of course possible in combination within acceptable range in the context of the objects of the present invention.

Further, in the first embodiment through the fifth embodiment and the modified examples of the present invention described above, illustrations are given by providing examples in which embodiments of the present invention are applied to a blood pressure monitor that can measure the maximum blood pressure and the minimum blood pressure and a blood pressure monitor cuff that is equipped therewith. However, embodiments of the present invention of course can apply to a sphygmomanometer that can measure other blood pressure information other than the maximum blood pressure and minimum blood pressure (for example, the mean blood pressure, the pulse wave, the pulse, the augmentation index (AI) value and the like) and to a sphygmomanometer cuff that is equipped therewith.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A cuff of a sphygmomanometer comprising:
a fluid bladder connected to a main body of the sphygmomanometer via a tube; and
a band-shaped wrapping body that encloses the fluid bladder, the band-shaped wrapping body comprising two longitudinal sides and two lateral sides,
wherein, a weight section is provided along a longitudinal side of the band-shaped wrapping body that faces a peripheral end of a user's arm when the cuff is mounted to the arm in accordance with an expected method of use.

2. The cuff of the sphygmomanometer according to claim 1, wherein the weight section is provided in a plurality of numbers at regular intervals.

3. The cuff of the sphygmomanometer according to claim 1, wherein the cuff is formed in a cylindrical shape having a hollow part such that the user's arm can be inserted into the hollow part.

4. The cuff of the sphygmomanometer according to claim 2, wherein the cuff is formed in a cylindrical shape having a hollow part such that the user's arm can be inserted into the hollow part.

5. A cuff of a sphygmomanometer comprising:
a fluid bladder connected to a main body of the sphygmomanometer via a tube; and
a band-shaped wrapping body that encloses the fluid bladder,
wherein a cylindrical shell for inserting a user's arm is rotatably connected to the main body of the sphygmomanometer, and the band-shaped wrapping body is mounted in a hole of the cylindrical shell, and
wherein a weight section is provided at a portion of the band-shaped wrapping body that contacts a vertical bottom position of the cylindrical shell when the user's arm is inserted into a hollow part of the shell in accordance with an expected method of use.

6. A cuff of a sphygmomanometer comprising:
a fluid bladder connected to a main body of the sphygmomanometer via a tube; and
a band-shaped wrapping body that encloses the fluid bladder,
wherein a cylindrical shell is rotatably connected to the main body of the sphygmomanometer, the cylindrical shell comprising a handle provided at a vertical top portion thereof, and the band-shaped wrapping body is mounted in a hole of the cylindrical shell to form a hollow part for inserting a user's arm therein, and
wherein the band-shaped wrapping body is provided with a weight section at a bottom position of the cylindrical shell that is opposite to the position of the handle in the cylindrical shell when a user's arm is inserted into the hollow part of the shell in accordance with an expected method of use.

7. A sphygmomanometer comprising:
a main body;
a cylindrical shell comprising a hole, the shell rotatably connected to the main body; and
a cuff stored in the hole of the shell, the cuff forming a hollow part for inserting a user's arm therein,
wherein the cuff comprises a weight section at a vertical bottom position of the user's arm when the user's arm is inserted into the hollow part of the cuff stored in the hole of the cylindrical shell in accordance with an expected method of use.

8. A sphygmomanometer comprising:
a main body;
a cylindrical shell that is rotatably connected to the main body, the shell comprising a handle at a vertical top position thereof; and
a cuff stored in a hole of the shell to form a hollow part for inserting a user's arm therein,
wherein the cuff comprises a weight section at a vertical bottom position of the cylindrical shell that is opposite to the handle of the cylindrical shell when the user's arm is inserted into the hollow part of the cylindrical shell in accordance with an expected method of use.
